(19) United States
(12) Patent Application Publication
(10) Pub. No.: US 2023/0146427 A1
(43) Pub. Date: May 11, 2023

(54) BIOFABRICATION AND MICROBIAL CELLULOSE BIOTEXTILE

(71) Applicants: Theanne SCHIROS, New York, NY (US); Helen H. LU, New York, NY (US); Romare M. ANTROBUS, New York, NY (US); Christan JOSEPH, New York, NY (US); Adrian M. CHITU, New York, NY (US); Shanece ESDAILLE, New York, NY (US); Susanne GOETZ, New York, NY (US); Anne VERPLOEGH CHASSÉ, New York, NY (US); ESPOSITO DANIELLA, New York, NY (US); Arianna WONG, New York, NY (US); Dylon SHEPELSKY, New York, NY (US)

(72) Inventors: Theanne Schiros, New York, NY (US); Helen H. Lu, New York, NY (US); Romare M. Antrobus, New York, NY (US); Christian Joseph, New York, NY (US); Adrian M. Chitu, New York, NY (US); Shanece Esdaille, New York, NY (US); Susanne Goetz, New York, NY (US); Anne Marika Verploegh Chassé, New York, NY (US); Daniella Esposito, New York, NY (US); Arianna Wong, New York, NY (US); Dylon Shepelsky, New York, NY (US)

(21) Appl. No.: 17/793,030

(22) PCT Filed: Jan. 14, 2021
(86) PCT No.: PCT/US2021/013518

§ 371 Date: Jul. 14, 2022

Related U.S. Application Data
(60) Provisional application No. 62/960,775, filed on Jan. 14, 2020.

(51) Int. Cl. D06M 16/00; B82Y 5/00

(52) U.S. Cl. CPC D06M 16/003; B82Y 5/00; D06M 2101/06

(57) ABSTRACT

A biomaterial may be produced by employing bacterial nanocellulose which are biologically functional and can be enhanced via an enzyme tanning treatment, to form a functional biotextile that has improved functionality and various desirable and/or enhanced properties.

This invention was made with government support under grant number DMR-1420634 awarded by the National Science Foundation (NSF) Division of Materials Research (DMR), Materials Research Science and Engineering Center. The government has certain rights in the invention.

### TECHNICAL FIELDS

An aspect of this disclosure pertains to biosynthesized bacterial cellulose.

Another aspect pertains to, inter alia, biofabrication methods, such as employing microbial cellulose.

In addition, another aspect pertains to fabrics and knits for garments and interiors formed from such biomaterials.

### BACKGROUND

An increased public awareness of fashion's environmental impact is driving the industry towards sustainable business models; industry leaders (including ADIDAS, LVMH and Inditex) are members of the Sustainable Apparel Coalition and Global Fashion Agenda, measuring their environmental and social labor impacts across the value chain in addition to working with policy makers to develop a wider framework for a circular fashion system. With an effort to reduce their environmental footprint, brands are actively seeking sustainable raw material alternatives to traditional fibers.

The fashion industry is one of the biggest contributors to climate change (1.2 billion tons of CO2 emissions per year), textile finishing and dye pollution and the single largest source of microplastic pollution globally [“The price of fast fashion”, Nature Clim Change 8, 1 (2018); DeFalco et al. Scientific Reports, (2019) 9:6633]. Machine washing of synthetic textiles is responsible for 23% of micro plastic pollution in the ocean. As such, textile production, finishing and end of life impacts are a major threat to biodiversity, an ecosystem's greatest resilience to the impacts of climate change.

The textile industry is also one of the most chemically intensive and ecotoxic industries on earth, and the second largest source of industrial water pollution (after agriculture), both in terms of the volume generated and toxicity of effluents [Sen S, Demirer GN. Water Research 2003; 37 (8) 1868-1878; Ben Mansour H, et al. Environmental science and pollution research international (2012); DOI 10.1007/s11356-012-0802-7; Rita Kant, Natural Science, 4, 1(2012), 17027]. Textile dyeing and finishing accounts for 20% of global water waste (R. Kirchain, et al. Sustainable Apparel Materials/Materials Systems Laboratory (2015)). Tannery effluents are ranked as the highest pollutants among all industrial wastes, due to the large volume of highly colored compounds, sodium chloride and sulphate, various organic and inorganic substances, toxic metallic compounds, different types of tanning materials which are biologically oxidizable, and large quantities of putrefying suspended matter [Akan et al. 2007; Khan et al. 1999].

They are especially large contributors of chromium pollution, toxic, mutagenic, carcinogenic, and teratogenic. In Hazaribagh (a particularly large tanning region of Bangladesh with over 200 tanneries), for example, it is estimated that 7.7 million liters of wastewater and 88 million tons of solid waste are disposed of annually. In India alone about 2000-3000 tons of chromium escapes into the environment annually from tannery industries, with chromium concentrations ranging between 2000 and 5000 mg/l in the aqueous effluent compared to the recommended permissible discharge limits of 2 mg/l [M. M. Altaf, F. Masood, and A. Malik, Turkish Journal of Biology, Vol. 32, 1-8, 2008]. These pollutants are responsible for the contamination of all nearby surface and groundwater systems with severely high levels of chromium [Bhuiyan, M. A. H., et al. Environmental Monitoring and Assessment, 175, 1-4 (2010): 633-649].

With growing demand for sustainable raw materials, many alternatives have been introduced to the marketplace, including recycled PET, sustainably produced regenerated cellulosic fibers, recycled wool, and emerging biomaterials [Bolt Threads, Tandem Repeat, Mango Materials, etc.]. However, many biomaterials still rely on conventional textile processes, which can be toxic and water intensive. Textile Exchange estimates that biomaterials economy has the potential to mitigate 2.5 million tons of CO2 (2020). Fiber alternatives, such as recycled polyester is heavily processed, relies on synthetic dyes, is not biodegradable, and releases microplastics.

### BRIEF SUMMARY

The inventors have devised methodologies for biofabricating scalable and functional, non-toxic biomaterials that have flexible functionality and can be integrated in a closed loop life cycle.

In this disclosure, a method is described for biofabricating a biotextile material, the method comprising:

(a) obtaining bacterial cellulose nanofibers;

(b) applying to the bacterial cellulose nanofibers a tanning treatment employing a phospholipid emulsion comprising lecithin; and

(c) processing the cellulose nanofibers into a 3D shape.

Also in this disclosure, a biofabrication method is described for forming a biotextile material, the method comprising:

(a) obtaining microbial cellulose nanofibers; and

(b) applying a phospholipid emulsion comprising lecithin to the microbial cellulose nanofibers; and

(c) subjecting the microbial cellulose nanofibers to an aldehyde treatment.

Still further in this disclosure, a composition is described of a biotextile material comprising biologically functional microbial cellulose nanofibers constituted of biologically functional microbial nanocellulose to which lecithin tanning has been applied, the lecithin tanning applied to the microbial cellulose nanofibers imparting an improved flame retardance property to the biotextile material.

Various other inventive aspects can be integrated or employed, as discussed infra.

### BRIEF DESCRIPTION OF THE DRAWINGS

FIG. 1: Biomaterial Design Criteria: diagram of design criteria for materials that can mitigate the environmental impacts of the textile industry.

FIG. 2A: Biochemical pathway for the production of bacterial cellulose. FIG. 2B: Biosynthesis of bacterial nanocellulose in liquid culture media and SEM image showing nanocellulose fibers.

FIG. 3A: Images of bacterial cellulose before (top panel) and after (bottom panel) being submerged in Di-H₂O for 24 hours. FIG. 3B: Image of contact angle of water droplet on as grown BC; tanned and smoked BC and smoked BC. FIG. 3C: Swell tests: percent change of mass after 24 hours in Di-H₂O. FIG. 3D: Graph of contact angle of water droplet on as grown BC; tanned and smoked BC and smoked BC. FIG. 3E: Swell tests: percent change of volume (axial swell) after 24 hours in Di-H₂O. FIG. 3F: Absorbance data showing the time in seconds for a water droplet shown in FIG. 3B to fully absorb into the materials (AATCC absorbency of textiles test AATCC TM79). Time greater than 300 seconds indicates a water resistance textile. Aldehyde treated BC (smoked) absorbed water at the slowest rate (FIG. 3F), where it took 3,481-4,200 seconds to fully absorb one drop of deionized water, with the higher absorption observed for thinner BC biofilms, with more visibly separated layers, and the upper end for thicker denser samples. The LT enzyme tanned absorbed water at the quickest rate (1,347-1,394 seconds). The absorption time of the samples that were both aldehyde treated and enzyme tanned were comparable to enzyme treatment alone (1,517 vs 1,590 seconds). The absorption time of the control samples that were for the control was less than that of the purely aldehyde treated samples but more than that of the enzyme and aldehyde treated BC (2,776-2,691 seconds). Aldehyde tanned BC consistently had the highest contact angle (FIG. 3B and FIG. 3D). These findings demonstrate the versatility of bacterial cellulose, and how, depending on its preparation, it has a wide range of applications such as water repellent textile or water-absorbent gauze or bandage.

FIG. 4A and FIG. 4B: Degree of swelling in BC samples. FIG. 4A: Degree of swelling of microbial cellulose samples throughout 24 hours for as grown (BC), enzyme tanned (LT), and BC sample purified in 1M NaOH (DC). FIG. 4B: Trial 2 swell tests for biosynthesis trial under the same cultivation conditions as in FIG. 3.

FIG. 5A: Tensile testing of as grown bacterial cellulose biotextile (BC); compared to enzyme tanned (BT, LT) and enzyme and aldehyde treated BC post-production (BTS, LTS). Post-production enzyme treatment increases the strength and flexibility of BC biotextiles relative to as grown BC. Aldehyde treatment (exposure to a mix of hydrocarbons from wood smoke) reduces the maximum strength but preserves the flexibility of the material, and improves hydrophobicity (see FIG. 4A and FIG. 4B). FIG. 5B: Comparison of enzyme treated BC (LTBC), as grown BC biotextiles with cotton and leather. Tanning increases both strength and flexibility. Smoking decreases strength but increases water contact angle and thermal stability.

FIG. 6A and FIG. 6B: Mechanical properties of microbial cellulose samples. FIG. 6A: Stress-strain curves for decellularized as grown control and lecithin tanned samples (n=1). FIG. 6B: Young's modulus, yield strength, toughness, resilience, and max strength calculations for decellularized as grown control (n=2) and lecithin tanned samples (n=3).

FIG. 7A: X-ray diffraction data comparison for as grown (control) BC and lecithin tanned, smoked and lecithin tanned and smoked BC samples. All curves show the characteristic reflections for BC. The peaks shift to higher angle (larger d-spacing/lattice parameter) and lower angle (larger d-spacing) for the smoked samples indicates a decrease in crystallinity after the aldehyde treatment, consistent with the lower maximum tensile strength (FIG. 5A and FIG. 5B). FIG. 7B: Difference in peak position disappears after LT BC sample is annealed, indicated the change in lattice spacing is due to a difference in hydration state, which underlies the change in mechanical properties.

FIG. 8: Thermogravimetric analysis further confirms that the change in mechanical properties and lattice parameter in BC after tanning is due to the amount of moisture in the lattice unit. George et al. and Auta et al. report that the moisture is removed from BC beginning under 100 degrees Celsius. As the lecithin tanned sample experiences the largest drop in mass in this segment of heating FIG. 8, it can be concluded that the lecithin tanned samples had the largest amount of moisture to begin with. The DTG diagram of the original BC shows a three-step decomposition process: the first step was up to about 150° C. and was attributed to the release of free and bound water and the main degradation step including depolymerization, dehydration, and decomposition of the cyclic structures was observed from about 200 to 400° C. In this main decomposition stage, about 60% of the mass of the samples was lost and about 50% of the lecithin tanned samples. This is significantly less than the mass lost by BC in other reports of the original BC was lost (Frone et al. Coatings 2018, 8, 221; doi: 10.3390/coatings806022). The shoulder observed on the right side of the main peak corresponds to the pyrolysis of cellulose (Rosa, J. R. et al. Cellulose, 2014, 21, 1361-1368). The charred residue obtained from the BC samples ranged from 5 to 20% with the aldehyde treated (smoked) samples at the lower end and the enzyme tanned samples at the higher end of this range. In FIG. 8, the moisture drop among the various samples indicates that smoking might evaporate any moisture that was retained by the tanning process. It is also possible that smoking can chemically change the environment of the lattice unit to be less hydrophilic. In a study conducted by Artigo et al., greater moisture content in cellulose as evidenced by TGA was attributed to a greater concentration of hydroxyl groups. Potentially, smoking could be changing these hydroxyl groups into a different functional group giving the lattice unit a lower affinity for water molecules. The gradual weight loss over the wide temperature range was attributed to the good thermal stability of BC (inflection temperature >320° C. (FIG. 9), but physical and chemical treatments may influence its thermal behavior (Frone et al. Coatings 2018, 8, 221; doi: 10.3390/coatings806022).

FIG. 9: TGA inflection temperature shows high thermal stability (T>320° C.) for BC. The thermal stability is affected and can be tuned with surface treatment and dehydration/hydration cycles (FIG. 9, fifth bar). Method: as grown control samples (n=5) were rehydrated via swell test for 24 hours, followed by drying at room temperature for 24 hours TGA shows that after rehydration and drying cellulose degraded at lower temperature similar to lecithin tanned samples. Further, the chemically induced change in lattice parameter may contribute to the change in mechanical properties shown in FIGS. 5A-B and FIGS. 6A-B in multiple ways: (1) Moisture content has been shown to increase the fibril-fibril bonding in hydrated XG cellulose (Prakobna et al.); (2) Hydroxyl groups from load baring network like structures that result in increased mechanical performance (George et al.). If treatment increases hydroxyl groups then it should have positive effects on the strength of the biomaterial produced by the methodologies described herein; and (3) Lattice parameter may be affecting mechanical properties and if the lattice unit is holding more moisture then lattice parameter would be expected to change accordingly, as observed in XRD data (shown in FIG. 7). FTIR validates that an increase in hydration state for the enzyme tanned BC increases the number of OH available for intra- and intermolecular bonding; thereby affecting the cross-linking and consequently, the mechanical properties of the treated samples, specifically the increase in strength, toughness, and resilience of the tanned samples. FIG. 9 shows the FTIR spectra of as grown, purified (DC) and enzyme tanned BC before and after vacuum anneal treatment. As denoted by literature, a characteristic absorption band from bacterial cellulose is observed at 1163 cm⁻¹ becomes less intense and broader. Similar peaks are found in FIG. 12, which shows the FTIR spectra of lecithin tanned samples before and after vacuum anneal treatment. In the spectrum, the peak at 1737 cm⁻¹ is representative of vibrations from C═O groups in lecithin molecules (Zhang et al.). Lecithin does not attach to microbial cellulose by hydrogen binding or electrostatic interactions, so surface detachment can occur by rising the sample with water. Nonetheless, the enzyme treatment affects the local bond environment (see also XPS in FIG. 13); these chemical changes manifest in physical changes in the mechanical properties of BC.

FIG. 10: FTIR of as grown BC, purified (DC) and enzyme tanned BC before and after vacuum anneal at 80° C. for 24 hours.

FIGS. 11A-E: FTIR regions of as grown BC, purified (DC) and enzyme tanned BC before and after vacuum anneal at 80° C. for 24 hours. All peaks sharpen and increase intensity relative to as grown BC indicating a more well-defined bond environment and chemical interaction between the lecithin, phospholipids and BC. The OH peaks sharpen, consistent with the change in hydration state which affects the cross-linking and improves the mechanical properties, including strength and resilience of the treated BC. Peaks at 1737 and 1203.7 cm⁻¹ (FIG. 11B and FIG. 11C) can be assigned to the vibrations of CO and PO groups in lecithin molecules, respectively (R. Nirmala et al. Mater. Sci. Eng., C, 31 (2011), pp. 486-493; Y. A. Shchipunov, E. V. Shumilina, Mater. Sci. Eng. C, 3 (1995), pp. 43-50), while the series of sharp peaks between 2853 cm⁻¹ and 3000.6 cm⁻¹ (FIG. 11A) are assigned to carbon-hydrogen (CH) band (N. Zhu, F. Z. Cui, K. Hu, L. Zhu, J. Biomed. Mater. Res., 82A (2007), pp. 455-461; Y. Wan, g. Zuo, C. Liu, X. Li, F. He, K. Ren, H. Luo, Polym. Adv. Tech., 22 (2011), pp. 2659-2664). The band from 2850 to 2950 cm⁻¹ is attributed to asymmetric and symmetric C—H stretching vibrations of methyl and methylene groups (FIG. 11A). This band is broad and fairly weak for as grown BC (2923.4, 2896.2 and 2853.4 cm⁻¹), but the band sharpens and increases in intensity for the lecithin tanned BC, and a weak but sharp C—H stretch appears at 3000.6 cm⁻¹ which is not observed for as grown BC. This suggests that the exocyclic CH₂ group may be involved in physical or chemical interactions during the treatments. Changes are also observed at 1428, 1370, and 1316 cm⁻¹, which are related to the C—H deformation (FIG. 11C). In the spectrum of BC, the typical broad peak at 3600-3300 cm⁻¹ corresponding to OH stretching band and physiosorbed moisture is observed (J. Wang et al. Mater. Sci. Eng., C, 32 (2012), pp. 536-541; C. R. Nirmala et al. Mater. Sci. Eng., C, 31 (2011), pp. 486-493). After vacuum anneal this band broadens and decreases in intensity while the C—H band (2850-2950 cm⁻¹) sharpens (FIG. 11A). In addition, the typical absorption bands at 1173 and 1130 cm⁻¹ (CO and COC stretching frequency band) belonging to the characteristic bands of BC (Wang et al. (2012); C. J. Grande et al. Mater. Sci. Eng. C, 29 (2009), pp. 1098-1104) are also observed. This suggests the presence of lecithin on the surface of BC fibers, likely through the weak physical adsorption, with the majority of changes attributed to the increased hydration state of lecithin tanned BC, increasing the concentration and availability of OH groups for intra- and intermolecular bonding. As there are no strong interactions between BC and lecithin, hydrogen bonding is expected to occur at the lecithin-BC interfaces and there may only be a weak electrostatic interaction. As such, lecithin is expected to detach from the BC surface of BC under flowing water. Lecithin is, however, likely to remain immobilized between the many layers of the BC biofilm after soak in the emulsion. The broad band from 3000 to 3500 cm⁻¹ shown in FIG. 11A corresponds to the hydroxyl groups involved in the intra-chain and inter-chain hydrogen bonds at 3410 cm⁻¹ and 3294-3300 cm⁻¹, respectively (Frone et al. Surface Treatment of Bacterial Cellulose in Mild, Eco-Friendly Conditions, Coatings (2018), 8, 221; doi:10.3390/coatings806022. An increase in intensity and sharpening of this band in the case of the treated BC compared to the as grown BC points to the increase in OH available for bonding, and the increased hydrophilicity, in agreement with the contact angle measurements (less OH for intra- and intermolecular bonding, the more hydrophobic). New peaks, which are characteristic to the stretching vibration of C═O esters, appeared in the FTIR spectra of LT BC 1737 and 1730 cm⁻¹, respectively (FIG. 11C). These groups are attributed to the reaction of the carboxylic acids with the OH groups of cellulose (Nishino, T., Koltera, M., Suetsugu, M., Murakami, H., Urushihara, Y. “Acetylation of plant cellulose fiber in supercritical carbon dioxide.” Polymer, 2011, 52, 830-836; Kalia, S., Kaith, B. S., Sharma, S., Bhardwaj, B. “Mechanical properties of flax-g-poly(methyl acrylate) reinforced phenolic composites.” Fiber. Polym. 2008, 9, 416-422); see also XPS in FIG. 13. Increase in intensity and sharpening of C═C stretch bands at 1644.6 cm⁻¹ (FIG. 11B), 1246.9 cm⁻¹ (FIG. 11C) and 984 cm⁻¹ (FIG. 11D) and an N—H peak at 1531.7 cm⁻¹ indicate a coupling between the lecithin and the cellulose polymer, consistent with a more well defined molecular bond environment underlying the change in cross-linking responsible for the improved mechanical properties of the treated BC.

FIG. 12: FTIR data for purified BC (DC) before and after vacuum anneal at 80° C. for 24 hours. The OH band at 3410 cm⁻¹, 3294-3400 cm⁻¹ and 1335 cm⁻¹ decrease while the C—H symmetric and asymmetric stretch modes at 2850-2950 cm⁻¹ sharpen and increase after water is removed from the sample and hydroxyl groups involved in the intra-chain and inter-chain hydrogen bonds within cellulose: O3H . . . O5 intramolecular hydrogen bond, as well as physiosorbed water, are removed.

FIG. 13: X-ray photoelectron spectroscopy (XPS) core level data for as grown BC and enzyme tanned (LT) after vacuum anneal at 75° C. for 24 hours to remove residual water and prevent outgassing in the UHV chamber. The data is an average of a minimum of n=3. The C is data shows the increase in surface C—O—C and C—H bonding, as well as carbon in the form of —COOR groups at the surface (carboxylic acids, esters and anhydrides), in agreement with the FTIR data (FIG. 10 and FIGS. 11A-E). The P 2p data shows phosphorous is introduced to the material by the tanning treatment, consistent with the appearance of an FTIR peak due to P—O vibrations at 1203.7 cm⁻¹ wave number in FIG. 11C. A dilute concentration of nitrogen (<1%) is also observed in the LT tanned BC. The change in line shape an shift of the O 1s core level peak to higher binding energy for the lecithin tanned BC is attributed to an increase in C—O, O—C—O, C═O and —COOR groups at the surface of the treated sample.

FIGS. 14A-F: Quantification of SEM images for microbial nanocellulose biofilms, FIG. 14A: as grown (BC); FIG. 14B: after 24 hours in lecithin emulsion (LT); FIG. 14C: after aldehyde treatment (smoked); FIG. 14D: lecithin tanned followed by smoke treatment; FIG. 14E: fiber diameter analysis (n=100) shows a decrease in fiber diameter for enzyme (LT) and aldehyde (smoked) treated BC and a slight increase in the nanofiber diameter when the two processes are combined (LTS). The increased nanofiber diameter for the LTS samples may suggest that the aldehyde treatment seals in the enzyme tanning so the fibers are coated and the lecithin does not rinse off; FIG. 14F: SEM images for microbial nanocellulose biofilms.

FIG. 15: SEM/EDAX confirms the phosphorous concentration increases with enzyme treatment, consistent with XPS data (FIG. 13).

FIGS. 16A-C: Flame testing. The inventors flame tested as grown and treated BC biotextiles using ASTM D1230-94, the 45° Flame Test-Standard Test Method for Flammability of Apparel Textiles. FIG. 16A: Under a 2740° F. flame, such tanned and smoked BC biotextile does not ignite and deflects the flame from the surface so it does not propagate along the biomaterial. FIG. 16B: Image of biotextile heated to an ash with the same flame. FIG. 16C: Image of same biotextile as FIG. 16B, showing the enzyme tanned and smoked BC can rehydrate within in hour.

FIG. 17: Clips from a video documenting flame testing of BC biotextiles using ASTM D1230-94, the 45° Flame Test-Standard Test Method for Flammability of Apparel Textiles. Under a 2740° F. flame, BC biotextile deflects the flame from the surface so it does not propagate along the biomaterial. The phenomenon is attributed to its nano-to-micro- to macro-scale properties, but is mostly due to the layered nature of a biofilm. The dense nanofiber mesh, crystalline microstructure and layered macrostructure all limit the availability of the requisite oxygen and heat needed to ignite the material and sustain a flame. The presence of phosphate groups, with known flame retardant properties (J. Tebby, Comprehensive Heterocyclic Chemistry II, Volume 8 (1996), 863-888), and increased hydration state of the tanned material (FIGS. 7A-B and FIG. 8) also enhance the inherent flame retardance of BC biotextiles. Shown here is a metal wire melting under the heat of the 2740° F. while the layered structure of the BC deflects the flame and traps hot gas between the layers. Smoking also inhibits the flammability of the nanocellulose fabric by charring the outermost surface layer.

FIG. 18: Life cycle impact assessment using Ecolnvent software shows the lecithin tanned BC (bacterial cellulose/microbial nanocellulose) has a 65-99% lower carbon footprint than chrome tanned animal (cow) leather, depending on the finishing chemicals used and at minimum of a 99% reduction in carcinogenic chemicals introduced to the environment.

FIG. 19: A significant percentage of the impacts from the BC biotextile fabrication is the polyethylene cultivation vessels. This could be reduced by utilizing glass or recycled stainless steel cultivation vessels.

FIG. 20: Diagram showing the potential of microbial biosynthesis and processing based in green chemistry to create minimal waste products with a closed loop life cycle (left). The right panel of the diagram sows scalable production of microbial nanocellulose from a variety of carbon sources, including fructose and sucrose (beakers A and B, respectively) grown to the shape of the vessel for zero waste patterning of a bag, and green chemistry processing-lecithin tanning and natural dyes to create low impact, minimal waste products, such as our biotexile prototypes, that meet design criteria of performance and biodegradability for a regenerative, circular materials economy.

FIGS. 21A-C: Physiochemical Characterization of Microbial Cellulose Textiles. FIG. 21A: SEM micrographs; FIG. 21B: tensile properties; FIG. 21C: XRD data; for as grown microbial cellulose (MC) and microbial-treated by lecithin (LTS) and/or smoke tanning (S, LTS).

FIGS. 22A-B: X-ray photoelectron spectroscopy (XPS) (FIG. 22A) Fourier Transform Infrared (FTIR) spectra (FIG. 22B) of microbial cellulose textiles before and after lecithin tanning.

FIGS. 23A-D: Microbial Cellulose Textiles Exhibit Flame Retardance. FIG. 23A: Under a 2740° F. flame, the lecithin tanned microbial cellulose biotextile does not ignite and deflects the flame from the surface so it does not propagate along the biomaterial. FIG. 23B: Torched biotextiles remain intact under the surface ash. FIG. 23C: SEM micrograph of post-combustion residue. FIG. 23D: Thermogravimetric (TG) curves of MC and treated samples under nitrogen atmosphere shows the phospholipid treatment directs the combustion chemistry toward ash formation over the glucosan formation to promote flame retardance.

FIG. 24: Life Cycle Impact Assessment of microbial cellulose (MC) biotextiles relative to conventional textiles, including leather, and PU textile (polyurethane-coated polyester), and woven cotton. Cradle to gate assessment for the industrial manufacture of 1 m² of textiles shown compared to laboratory-scale production and processing of MC (biotextile 1), and biotextile processing from MC obtained as a fermentation by-product of commercial kombucha production.

FIGS. 25A-O: Microbial biotextiles with color from natural dyes, including: (FIG. 25A and FIG. 25B) madder root; (FIG. 25C) cochineal, (FIG. 25D and FIG. 25E) marigold; (FIG. 25F) as fabricated (tannins from tea); (FIG. 25G); as fabricated treated with a heated soy wax, pine resin and jojoba oil coating; (FIG. 25H) yellow onion skins; (FIG. 25I) chlorophyll; (FIG. 25J and FIG. 25K) indigo; (FIG. 25L) color removed by 0.5 M NaOH; (FIG. 25M) natural black created by chemical interaction of iron mordant, FeSO₄, obtained by soaking nails in equal parts white vinegar and water, with tannic acid (from black tea); (FIG. 25N) FeSO₄-tannic acid dark stripes on as fabricated MC; and (FIG. 25O) marigold flowers embedded between MC pellicle layers before drying showing design opportunities such as no sew seams affordable by the layered structure of the moldable biotextile; and design prototypes, including (FIG. 25P) sneakers in natural (as fabricated) color and an upcycled rubber sole; and (FIG. 25Q) naturally dyed, “bioleather” wallets; opaque black achieved using myrobalan, followed by logwood, and finally a 10 second immersion in FeSO₄ before the biotextile was rinsed in water, lecithin tanned and dried; semi-transparent white as in (FIG. 25L). See Methods for details.

FIG. 26: Mechanical tensile date (n=5) (bottom) for biotextile 2—microbial cellulose obtained as a by-product from commercial kombucha fermentation (top)—provided by OM Champagne Tea, Mount Kisco, N.Y., lecithin-tanned and dried.

FIG. 27A: In situ natural dye coloration of MC biotextiles, with using turmeric, cochineal and chlorophyll, and blue food coloring (left to right). FIG. 27B: Resulting MC biotextiles swatches after dehydration, including a transparent white obtained by immersion of MC in 0.5 M NaOH for one hour, followed by rinse in distilled water until reaching a neutral pH.

FIG. 28: Initial frame from a time lapse of microbial cellulose biosynthesis over 21 days at 23° C. using: a) sucrose (left); and b) fructose (right) as carbon sources shows tunable and scalable biosynthesis and the ability of G. xylinum to utilize a variety of nutrient sources in its carbon metabolism. See https://vimeo.com/492333189.

FIG. 29: Flow chart for a method for biofabricating a biotextile material, according to an embodiment.

FIG. 30: Flow chart for a biofabrication method for forming a biotextile material, according to another embodiment.

### DETAILED DESCRIPTION

Disclosed herein are enzyme and aldehyde tanning methods which can control the hydration state and improve the flexibility, strength, water resistance and hand feel of bacterial cellulose biotextiles and “bioleather” without addition of other fibers or chemically intensive processing. The scalability, low impact and nontoxicity of enzyme and aldehyde tanned and naturally dyed bacterial cellulose (BC) bioleather and biotextiles position it as a viable sustainable, biodegradable alternative for apparel, interiors, displays, and fire-retardant emergency tents and protective apparel, that can meet consumer demand and industry demand without the toxicity, and environmental and human health impact of conventional textiles production and finishing.

Biocomposites can be created to further expand and improve material properties. The BC biotextiles are highly flame retardant, grown to shape from waste streams, and color can be achieved through the cultivation media itself, without the water demands or toxicity of dip dye methods.

Textiles, including apparel and interiors (furniture, displays) and tents, that employ flame retardant materials, have historically been used for large scale and/or short term events. In the United States alone, landfills receive 600,000 tons of waste from trade shows alone. This makes the trade show industry the second largest producer of landfill waste next to the construction industry (“Exploring Environmentally Friendly Trade Show Options.” Derse. Sep. 8, 2016). Many display materials have a large environmental impact which is exacerbated by the single-use purpose of the displays. One of the major public health and environmental concerns that these displays raise is their mandatory use of flame retardants. Commercial flame retardants have been tied to a number of health problems, especially in young children. These conditions include stunted neurodevelopment, thyroid disorders, problems with motor performance (coordination, fine motor skills), cognition (intelligence, visual perception, visuomotor integration, inhibitory control, verbal memory, and attention), and behavior. In order to address the environmental and public health impacts of trade shows, safer and more sustainable materials must be implemented.

The inventors have biosynthesized bacterial cellulose (BC) and developed tanning techniques, including lecithin/phosphatidylcholine enzyme and aldehyde treatment, to overcome inherent material challenges, including water resistance, strength and flexibility, that have inhibited direct translation of the biomaterial to a range of textile applications. Using a class of aerobic nonpathogenic bacteria that secrete very pure natural exopolysaccharide known as bacterial cellulose (BC) under special culturing conditions, the most commonly used being Gluconacetobacter xylinus (reclassified from Acetobacter xylinum), and adjusting the culture conditions and composition, the inventors produced BC-based biomaterials in flexible form factors, including non-wovens, wovens, fabric sheets, fibers and knits suitable for sustainable, flame retardant textile applications for insulating interiors, displays, and garments. The inventors have screen printed functional circuits on BC biotextiles and incorporated modular lighting and sensing electronic components to demonstrate the viability of this application. The extreme flame retardance of the BC biotextiles treated as described herein (i.e., enzyme tanning and aldehyde surface treatment) present it as a low cost, scalable, non-toxic option for flash tents and emergency fire fighter clothing.

Phosphatidylcholine (lecithin) is utilized as a byproduct from the edible oil industry, and associated phospholipids, fatty acids and choline from lecithin-containing agricultural materials and oils (eggs, sunflower, rapeseed, soybean) in animal and plant tissues as an enzyme tanning treatment to create a functional biotextile from bacterial nanocellulose. The enzyme treatment can be followed by an aldehyde treatment to seal the tanning treatment and add water resistance to the BC biotextile. Culture-media composition and conditions during biosynthesis and post-production aldehyde treatments are tailored to control the hydration state and thereby the material properties to create a “bioleather” for textile applications. The treatment is a novel adaptation of ancient techniques used to tan and smoke hides with oils and enzymes obtained from mammalian brain and aldehyde treatment from wood smoke to produce soft and supple leather, that stays pliable even after getting wet, applied to BC as a low impact, non-toxic, sustainable biotextile with a circular life cycle.

Gluconacetobacter xylinus, a gram-negative bacteria, is the most widely studied species of bacteria that produces cellulose. Under specific culturing conditions, the bacteria secrete cellulose in the form of a 3D network swollen gel (>90% water) of nano- and microfibrils (10-100 nm width), which coagulate into a thick mesh called a pellicle, which floats to air-culture media interface seeking oxygen. At the air-culture media interface, layer by layer biofilm growth produces a mat of cellulose (pellicle) up to several centimeters thick). BC presents unique properties including high mechanical strength, high crystallinity, high water holding capacity, biocompatibility and high porosity, and has been a type of nanostructured cellulose widely used as a biomaterial (Rajwade et al. 2015). The biofilm is composed of cellulose microfibrils and ˜97-99% water and can easily be manipulated according to the size and shape of the of the vessel used for cultivation. Microbial cellulose (MC) is a regenerating biomaterial which, depending on the thickness of biofilm and biosynthesis conditions, forms a thin paper-like sheet or a thick, leather-like fabric in its dehydrated stated. The hydrated MC biofilm can be easily molded to form 3-D shapes and layered and overlaid to form “seamless” seams as the biomaterial dehydrates and forms bonds to itself to form meshes of different fiber density and mechanical properties. It can also be blended into a slurry suitable for composite materials.

MC is an exopolysaccharide with unique structural and mechanical properties and is highly pure compared to plant cellulose. Microfibril formation, morphology, crystallization and mechanical properties of the biotextile can be altered by adjusting the external culturing conditions, such as temperature, solvent and concentration as well as the composition of the culture media (Applications of nanomaterials: advances and key technologies (Bhagyaraj), Duxford, Elsevier/Woodhead Publishing, (2018) 68-70).

MC nanofibers can by biosynthesized in a fermentation process by a Symbiotic Colony of Bacteria and Yeast (SCOBY) in culture media that may include tea, glucose from a variety of sources, and acetic acid. Bacterial cellulose producers can be extracted from a range of rotting fruit (B. E. Rangaswamy, et. Al. International Journal of Polymer Science, 2015 (2015), 280784), and the sugar can be obtained from food waste streams. Fermentation of waste and by-product streams from biodiesel and confectionery industries are also potential sources of efficient production of bacterial cellulose (E. Tsouko, et al. Int. J. Mol. Sci. 2015, 16, 14832). The ability to biosynthesize MC from sugar waste streams and biofabricate BC to shape allows for zero-waste pattern making and zero waste in the production phase for textile applications.

Bacterial cellulose is already being used for wound healing, as it is microscopically similar in structure to body tissue (Wojciech Czaja, et al., Biomaterials, 27, 2, 2006, 145-1510), as well as tissue cell cultures and a range of other biomedical and electrochemical applications. It also has potential as a low environmental impact, circular economy biotextile for a wide range of applications. However, BC has inherent materials challenges that limit direct translation into textile applications, including water resistance, strength and flexibility. For instance, as grown MC is hygroscopic, it would be anticipated to readily swell and dehydrate with ambient humidity, which has made it favorable for biomedical and electrochemical applications.

Enzyme and aldehyde treatments were used to produce a compostable BC biotextile with the requisite strength, flexibility and water resistance for textile applications, as well as extreme flame-retardance without use of petroleum-derived or synthetic chemicals. An emulsified solution of phospholipids and enzymes, derived from mammalian tissue and oils from seeds including, but not limited to, sunflower, cactus, and water, provide superior strength and flexibility to the materials as well and reduced hygroscopicity. Tannins, including catechol and pyrogallol, as well as plant oils (obtained from a wide range of plants, including sumac leaves or bark, including Quebracho, Chestnut or Mimosa), may be utilized as a “tanning” agent for MB biotextiles. Tannins also act as an anti-microbial agent during post-production textile use (the Gluconacetobacter xylinus bacteria used in the biosynthesis do not survive outside the culture media or in the dehydrated state of BC).

The emulsion can be added to the culture media used for BC biosynthesis, or used as a post-production treatment. As a post-production treatment, the hydrated or dehydrated BC biofilm is immersed in the emulsion for a minimum of 1 hour before being washed in DI water and then dried. The BC may also be introduced to the emulsion as a slurry. Once the BC has absorbed the liquid, an additional aldehyde treatment, such smoke tanning, in which BC biotextiles undergo prolonged exposure (10 minutes to 2 hours) to hydrocarbon-rich smoke, seals in the lecithin and oils. MC was placed 15 to 50 centimeters above the smoldering fire, during which the temperature was controlled (100-200 degrees F.) to optimize smoking and avoid charring.

The phospholipid/enzyme/oil concentration of the emulsion may be between 10-85 wt %. The amount of emulsion incorporated into the layered biomaterial can 5-50 wt %. The mass of the soaking emulsion for post-production treatment should be 20-300% percent of the mass of the BC biomaterial, though larger volumes can be used to scale the process to yardage of BC. “Tanning” of the BC bioleather can be achieved during biosynthesis, avoiding additional water use, space and time, with the emulsion 5-60% volume percent of the culture medium.

The breaking of unsaturated bonds in fatty acids during their degradation is also a source of aldehydes in low concentration, which help give the BC water resistance due to the hydrophobic nature of the fats, and provide a source or aldehyde tanning (C. L. Heth, Chemical Technology in Antiquity (2015), 181-196). The fatty emulsion, which can be high in myelin, is itself a source of aldehyde tanning over time.

The BC-tanning treatment more than doubled the strength and flexibility of the biomaterial, and increased its water resistance. Under a 2740° F. flame, our BC biotextile does not ignite- and deflects the flame from the surface so it does not propagate along the biomaterial. Moreover, when heated to an ash with the same flame, the enzyme tanned and smoked MC can rehydrate within an hour. Utilizing a low energy consuming organism to produce a flame retardant, biodegradable material to shape, opens an avenue to eliminate waste and toxicity, and to reduce carbon and water footprints throughout a BC-based product's circular life cycle. The Environmental Protection Agency found flame retardant chemicals in the systems of 95% of U.S. families, with rates higher in children, which have been linked to a myriad of health problems, including autoimmune diseases, learning disabilities, neurological and reproductive problems, birth defects, and possibly cancer (Gascon, et al. Environment International, 37, 3 (2011), 605-611).

Once treated, the BC biotextile is water resistant and durable. The treated MC compostable but the treatment enhances the durability and resistance to chemical degradation and decomposition, outside of a microbial rich environment such as a compost pile or bin at its useful end of life.

Dyeing and finishing (tanning) of textiles is one of the most chemically intensive industries on earth, and the second largest source of industrial water pollution (after agriculture), both in terms of the volume generated and toxicity of effluents (Sen S, Demirer GN. Water Research 2003; 37 (8) 1868-1878; Ben Mansour H, et. al Environmental science and pollution research international 2012; DOI 10.1007/s11356-012-0802-7; Rita Kant, Natural Science 4 No. 1(2012), Article ID:17027]). The enzyme and aldehyde treatment is a non-toxic alternative to chrome-based tanning (see Section V: Commercial Potential) of animal leather.

Further, coloration of BC fabric and fibers was achieved by adding a plant or mineral derived colorant to the culture media during biosynthesis, eliminating both the water usage and toxicity of conventional dip dye methods. We have identified a range of nontoxic natural dyes extracted from plants, minerals and insects that can be used without affecting the inherent chemistry or mechanical properties or the chemistry driving the biosynthesis of the MC textile, during biosynthesis and using post-production dip dye methods. Control over plant dye chemistry (pH, tannin concentration, etc.) and culture conditions can be used to adjust the mechanical properties of BC biotextiles.

Additional post-production processes can be applied to the BC biotextile to increase strength and water resistance. Controlled dehydration/hydration/dehydration cycles can be used to achieve an increased tensile strength and give different biotextile textures and hand feel.

The scalability, low impact and nontoxicity of enzyme tanned and naturally dyed BC biotextiles position it as a viable sustainable materials alternative for textiles, interiors, and the fashion industry at large, with a dramatically reduced environmental footprint relative to conventional textiles. Preliminary life cycle impact assessment (LCA) indicates that our lecithin-tanned BC “bioleather” has a 65-99% lower carbon footprint than chrome tanned animal (cow) leather, depending on the finishing chemicals used and at minimum of a 99% reduction in carcinogenic chemicals introduced to the environment.

In order to facilitate an understanding of the subject matter disclosed herein, each of the following terms, as used herein, shall have the meaning set forth below, except as expressly provided otherwise herein.

As used herein, “biofabrication” shall mean generation of materials or products from raw materials that comprise cells, are derived from cells, or are produced by cells.

As used herein, “biotextile material” shall mean any material used to create a textile, including but not limited to a fabric, film, etc., that is generated from a raw material that is comprised of or derived from cells. An example of a biotextile material includes biologically derived non-woven leather-alternative, also referred to as “bioleather,” for example, as generated from a bacterial cellulose.

As used herein, “cellulose” shall mean a polysaccharide consisting of a linear chain of multiple β(1-4) linked D-glucose units. Cellulose is generated by, for example, most plant cells or certain bacterial cells. Accordingly, cellulose generated by bacteria cells is interchangeably referred to as “bacterial cellulose” or “microbial cellulose.” As used herein “nanocellulose” shall mean a nano-structured cellulose, which may have a fibril width of several nanometers and range of lengths up to several micrometers (or more).

As used herein, the term “fibers” shall mean nanofibers of any substance that is significantly longer than it is wide. For example, fibers (or nanofibers) may be arranged or aggregated to form a mesh.

As used herein, the term “tanning” shall mean any process or treatment for preparing a material to produce a natural or biologically-derived leather-alternative product, e.g., “bioleather.” Tanning may comprise, for example, exposure of the material to enzymes, i.e., “enzyme tanning.”

As used herein, “aldehyde treatment” shall mean exposing a material to aldehydes, for example, by “smoking”, e.g., exposure to a mix of hydrocarbons from wood smoke.

As used herein, “emulsion” is a mixture of two or more liquids that are normally immiscible owing to liquid-liquid phase separation.

As used herein, “lecithin” is a generic term to designate any group of yellow-brownish fatty substances occurring in tissues, or otherwise produced from cells (e.g., plant, mammalian, etc.), which are amphiphilic. Lecithin may contain phospholipids, such as phosphatidylcholine.

As used herein, “phospholipids” are a class of lipids whose molecule has a hydrophilic “head” containing a phosphate group, and two hydrophobic “tails” derived from fatty acids, joined by an alcohol residue.

As used herein, “sunflower seed oil” is the non-volatile oil pressed from the seeds of a sunflower species, e.g., Helianthus annuus.

As used herein, “gram-negative bacteria” shall mean any bacteria that does not retain the crystal violet stain used in the gram-staining method of bacterial differentiation. Representative gram-negative bacteria include, for example, Gluconacetobacter xylinus, E. coli, etc.

As used herein, all numerical ranges provided are intended to expressly include at least the endpoints and all numbers that fall between the endpoints of ranges.

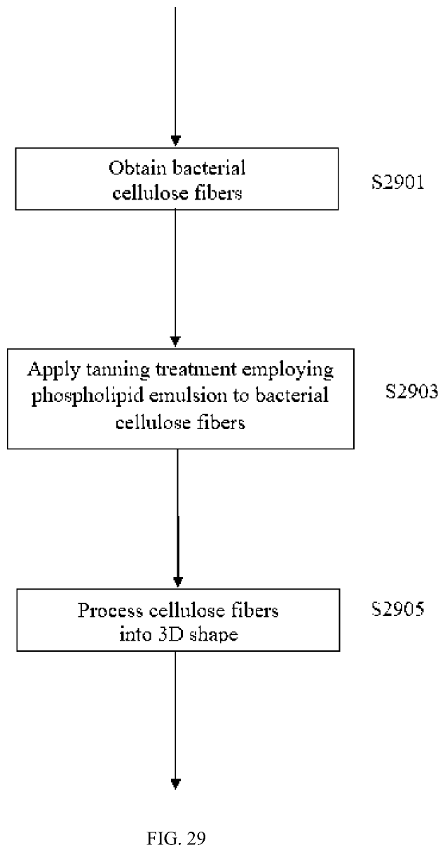

In this disclosure, a method is described for biofabricating a biotextile material (FIG. 29), the method comprising:

(a) obtaining bacterial cellulose nanofibers (S2901);

(b) applying to the bacterial cellulose nanofibers a tanning treatment employing a phospholipid emulsion comprising lecithin (S2903); and

(c) processing the cellulose nanofibers into a 3D shape (S2905).

In some embodiments, the method further comprises subjecting the bacterial cellulose nanofibers to an aldehyde treatment.

In some embodiments, the method further comprises subjecting the bacterial cellulose nanofibers to a coloration treatment.

In some embodiments, the phospholipid emulsion employed in the tanning treatment applied to the bacterial cellulose nanofibers in (b) further comprises one or more of sunflower seed oil or other phospholipids, fatty acids and choline. The phospholipid emulsion may be derived from, for example, plant or animal tissues including, but not limited to, mammalian organs such as brains, etc.

In some embodiments, the bacterial cellulose nanofibers are biologically functional bacterial cellulose nanofibrils produced extracellularly via microbial biosynthesis by living cells of gram-negative bacteria.

In some embodiments, the bacterial cellulose nanofibers are biologically functional and are formed from a culture containing living cells of Gluconacetobacter xylinus bacteria.

In some embodiments, the bacterial cellulose nanofibers are biologically functional and are molded into the 3D shape in (c).

In some embodiments, the bacterial cellulose nanofibers are arranged as a three-dimensional layered structure formed by a self-assembled network of biologically functional bacterial cellulose nanofibrils produced extracellularly via microbial biosynthesis by living cells of gram-negative bacteria.

In some embodiments, the bacterial cellulose nanofibers are arranged as the three-dimensional layered structure in a cultivation vessel having a shape corresponding to the 3D shape.

In some embodiments, the bacterial cellulose nanofibers are arranged as a three-dimensional network of unaligned biologically functional nanofibers in a cultivation vessel having a shape corresponding to the 3D shape, and the phospholipid emulsion is applied in (b) to the biologically functional microbial cellulose nanofibers in the cultivation vessel, the unaligned biologically functional nanofibers self-assembling into a three-dimensional structure corresponding to the 3D shape.

In some embodiments, the lecithin in the phospholipid emulsion applied to the bacterial cellulose nanofibers imparts an improved flame retardance property to the biotextile material.

In some embodiments, the improved flame retardance property of the biotextile material from the lecithin remains even after the aldehyde treatment of the microbial cellulose nanofibers.

In some embodiments, the bacterial cellulose nanofibers are obtained in (a) via biosynthesis, and the phospholipid emulsion comprising lecithin is applied in (b) to the bacterial cellulose nanofibers during the biosynthesis.

In some embodiments, the bacterial cellulose nanofibers are obtained in (a) via biosynthesis, and the phospholipid emulsion comprising lecithin is applied in (b) to the bacterial cellulose nanofibers after the biosynthesis.

In some embodiments, the cellulose nanofibers are processed into the 3D shape in (c) before, while or after the phospholipid emulsion comprising lecithin is applied in (b) to the bacterial cellulose nanofibers.

In some embodiments, the phospholipid emulsion comprising lecithin is applied in (b) to the bacterial cellulose nanofibers, at least partially at same time that the cellulose nanofibers are being processed into the 3D shape in (c).

In some embodiments, the method further comprises: processing the bacterial cellulose nanofibers obtained in (a) into a slurry; and depositing the slurry comprising the processed bacterial cellulose nanofibers into a cultivation vessel, the bacterial cellulose nanofibers in the slurry in the cultivation vessel being processed into the 3D shape in (c).

In some embodiments, the method further comprises: processing the bacterial cellulose nanofibers obtained in (a) into a slurry; and depositing the slurry comprising the processed bacterial cellulose nanofibers into a cultivation vessel, in (b) applying the phospholipid emulsion comprising lecithin to the bacterial cellulose nanofibers in the slurry in the cultivation vessel.

In some embodiments, the method further comprises: processing into a slurry the bacterial cellulose nanofibers to which the phospholipid emulsion comprising lecithin has been applied in (b); and depositing the slurry comprising the processed bacterial cellulose nanofibers into a cultivation vessel, the bacterial cellulose nanofibers in the slurry in the cultivation vessel being processed into the 3D shape in (c).

This disclosure also provides a biotextile product formed by the method for biofabricating a biotextile material described above.

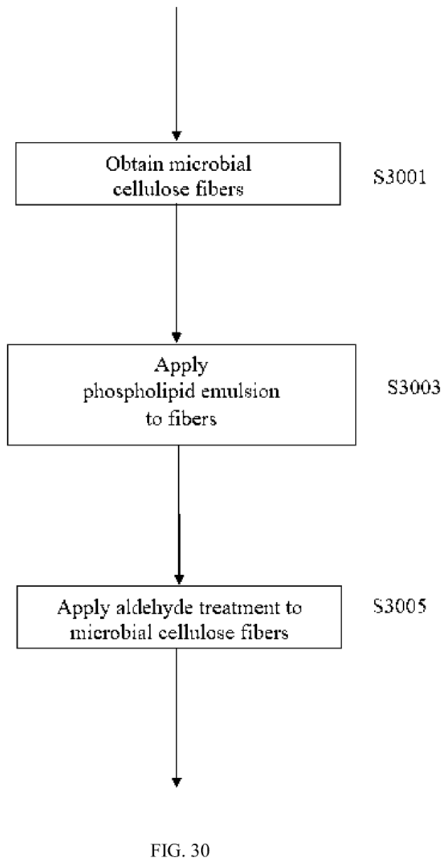

Also in this disclosure, a biofabrication method is described for forming a biotextile material (FIG. 30), the method comprising:
(a) obtaining microbial cellulose nanofibers (S3001);
(b) applying a phospholipid emulsion comprising lecithin to the microbial cellulose nanofibers (S3003); and
(c) subjecting the microbial cellulose nanofibers to an aldehyde treatment (S3005).

In some embodiments, the microbial cellulose nanofibers obtained in (a) are arranged as a three-dimensional layered structure formed by a self-assembled network of biologically functional microbial cellulose nanofibrils which are produced extracellularly via microbial biosynthesis by living cells of gram negative bacteria.

In some embodiments, the microbial cellulose nanofibers obtained in (a) are arranged in a cultivation vessel corresponding to a desired 3D shape of a biomaterial product, and the phospholipid emulsion is applied in (b) to the microbial cellulose nanofibers in the cultivation vessel.

In some embodiments, the method further comprises: forming biotextile including processing the microbial cellulose nanofibers into a 3D shape.

In some embodiments, the cellulose nanofibers are processed into the 3D shape before, while or after the phospholipid emulsion comprising lecithin is applied in (b) to the microbial cellulose nanofibers.

In some embodiments, the phospholipid emulsion comprising lecithin is applied in (b) to the bacterial cellulose nanofibers, at least partially at same time that the microbial cellulose nanofibers are being processed into the 3D shape.

In some embodiments, further comprising: processing the microbial cellulose nanofibers obtained in (a) into a slurry; and depositing the slurry comprising the processed microbial cellulose nanofibers into a cultivation vessel, the microbial cellulose nanofibers in the slurry in the cultivation vessel being processed into the 3D shape.

In some embodiments, further comprising: processing the microbial cellulose nanofibers obtained in (a) into a slurry; and depositing the slurry comprising the processed microbial cellulose nanofibers into a cultivation vessel, in (b) applying the phospholipid emulsion comprising lecithin to the microbial cellulose nanofibers in the slurry in the cultivation vessel.

In some embodiments, further comprising: processing into a slurry the microbial cellulose nanofibers to which the phospholipid emulsion comprising lecithin has been applied in (b); and depositing the slurry comprising the processed microbial cellulose nanofibers into a cultivation vessel, the microbial cellulose nanofibers in the slurry in the cultivation vessel being processed into the 3D shape.

In some embodiments, the method further comprises: subjecting the microbial cellulose nanofibers to a coloration treatment.

In some embodiments, the microbial cellulose nanofibers are biologically functional, and the emulsion applied to the microbial cellulose nanofibers in (b) further comprises phospholipids, fatty acids and choline.

In some embodiments, the lecithin in the phospholipid emulsion applied to the microbial cellulose nanofibers in (b) imparts an improved flame retardance property to the biotextile material.

In some embodiments, the improved flame retardance property of the biotextile material from the lecithin remains even after the aldehyde treatment of the microbial cellulose nanofibers.

In some embodiments, the microbial cellulose nanofibers are obtained in (a) via biosynthesis, and the phospholipid emulsion comprising lecithin is applied in (b) to the microbial cellulose nanofibers during the biosynthesis.

In some embodiments, the microbial cellulose nanofibers are obtained in (a) via biosynthesis, and the phospholipid emulsion comprising lecithin is applied in (b) to the microbial cellulose nanofibers after the biosynthesis.

This disclosure also provides a biotextile product formed by the biofabrication method for forming a biotextile material described above.

Still further in this disclosure, a composition is described of a biotextile material comprising biologically functional microbial cellulose nanofibers constituted of biologically functional microbial nanocellulose to which lecithin tanning has been applied, the lecithin tanning applied to the microbial cellulose nanofibers imparting an improved flame retardance property to the biotextile material.

In some embodiments, the biologically functional microbial cellulose nanofibers are arranged as a three-dimensional layered structure formed by a self-assembled network of biologically functional microbial cellulose nanofibrils which are produced extracellularly via microbial biosynthesis by living cells of gram negative bacteria.

Inspired by the complexity of nature and its robust regenerative potential, the inventors have harnessed microbial biosynthesis for the sustainable development of high performance, regenerative biotextiles. Biofabrication is a strategy to produce biologically functional products with structural organization from living cells, hybrid tissue constructs, and/or biomaterials. Microbial cellulose (MC), produced extracellularly by gram-negative bacteria such as Gluconacetobacter xylinus, exhibits unique properties, including a 3-D layered network of nanofibers with tunable self-assembly and robust tensile and shear mechanical properties. Drawing from a serendipitous synergy between ancient leather tanning techniques and the drive for biobased plasticizers, this disclosure describes a plant-based phospholipid processing technique that imbues MC with the requisite strength and ductility for regenerative, high-performance biotextiles, in addition to extreme flame retardance for widespread application in construction and insulating materials. Life cycle assessment shows that these biofabricated textiles have dramatically reduced environmental impacts relative to conventionally manufactured textiles, which are reliant on nonrenewable resources, petrochemicals, and energy and chemically intensive processes, as well as extreme water and land use demands. The translational potential of MC is tremendous, as the use of microbes to direct biomaterial formation in a bottom-up strategy that can strategically eliminate the toxicity and climate impacts of traditional manufacturing processes, as well as plastic pollution. Microbial cellulose is setting the basis for a ‘green’ materials economy, in which controllable microbial fermentation, post-synthesis modification, and subsequent biodegradability support the design of complex biomaterials with a sustainable and circular life cycle.

### EXPERIMENTAL DATA

### Experimental Set 1

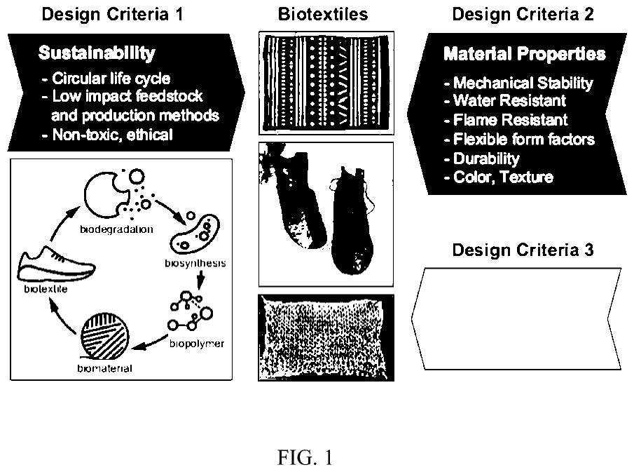

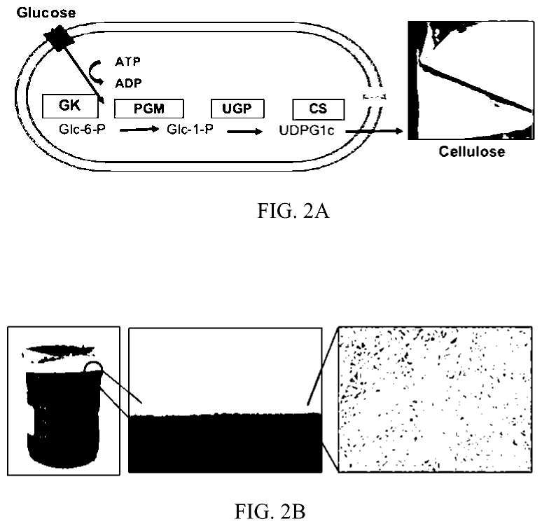

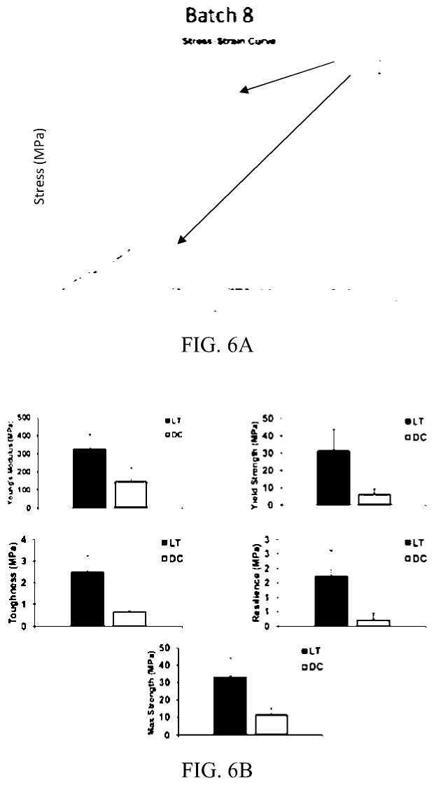

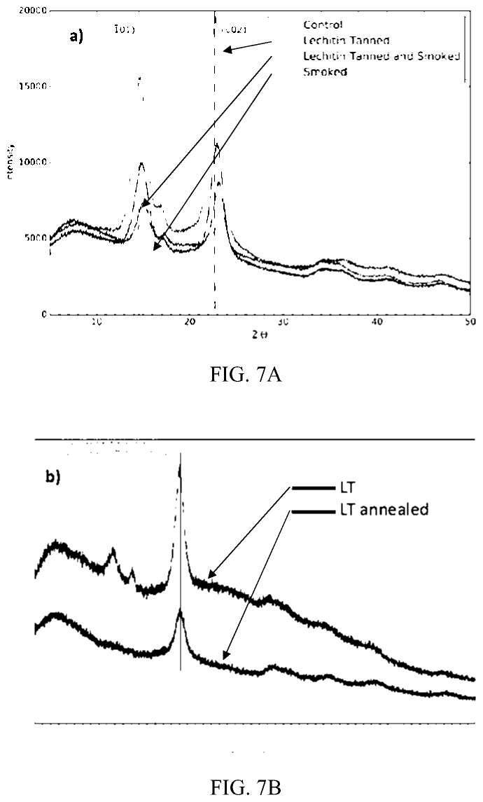

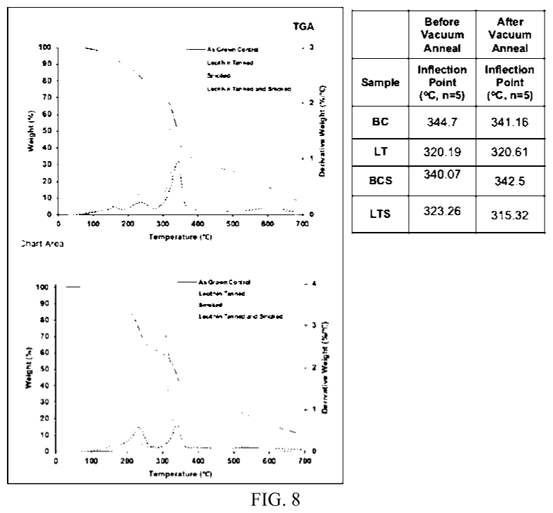

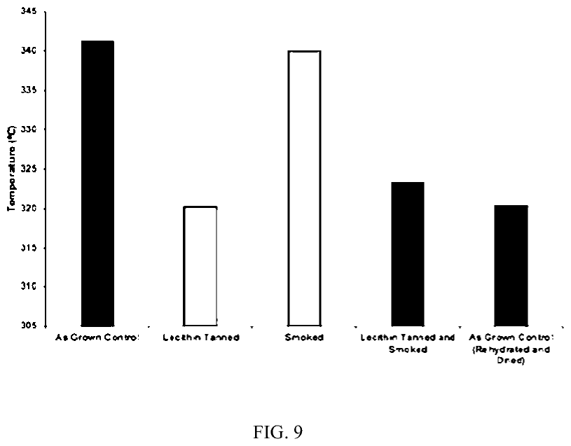

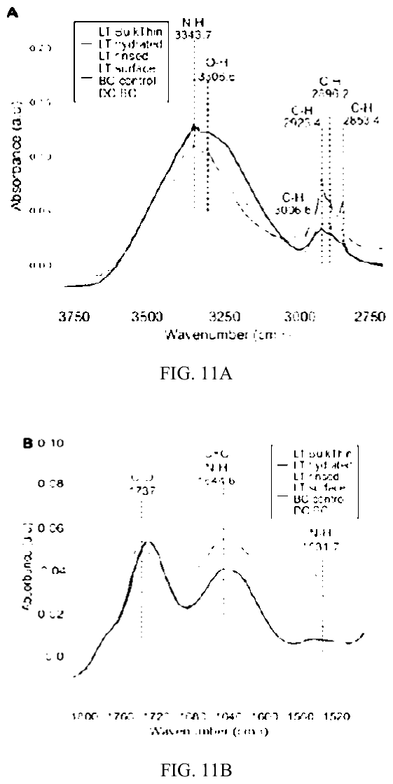

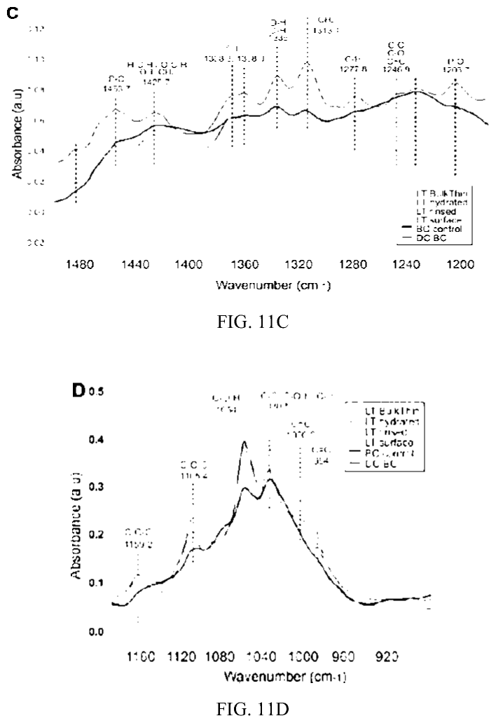

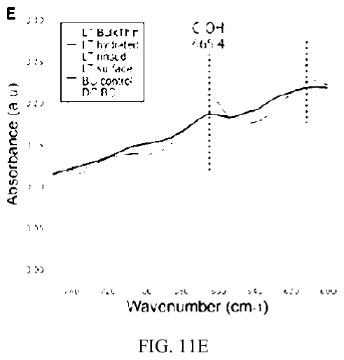

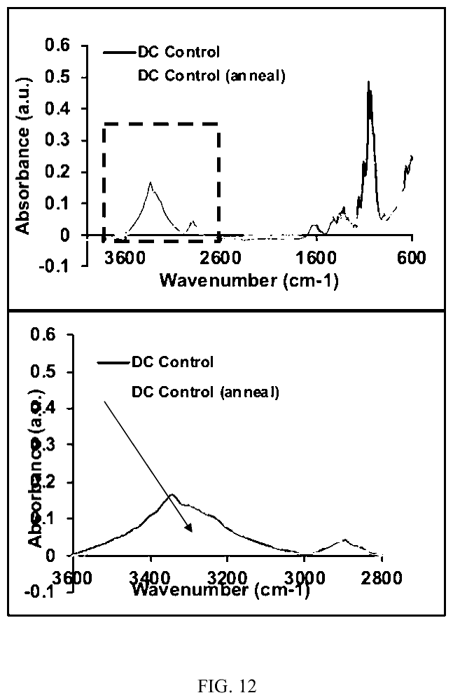

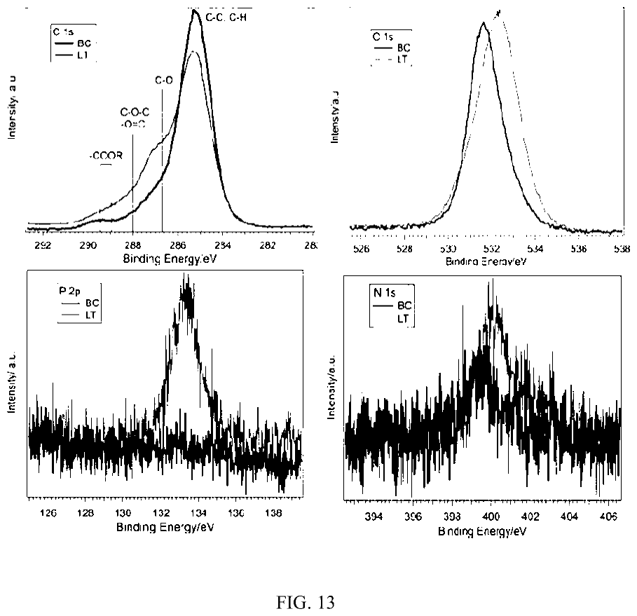

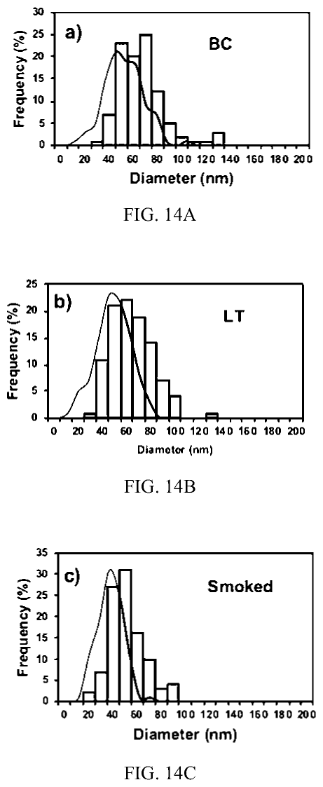

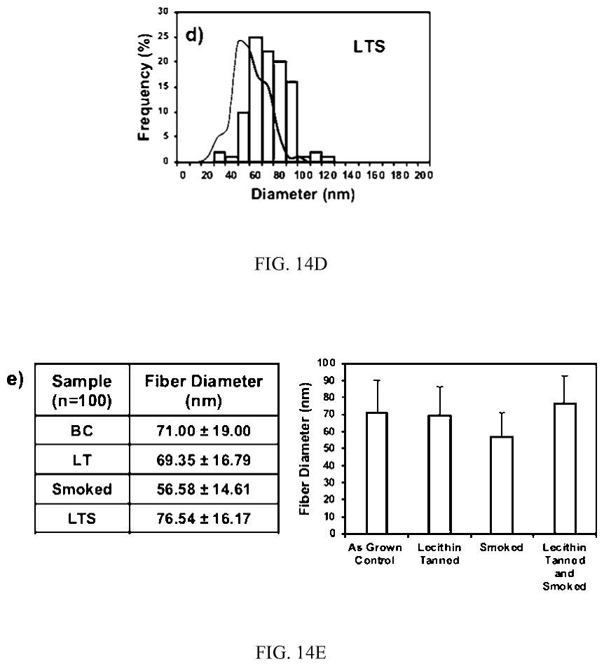

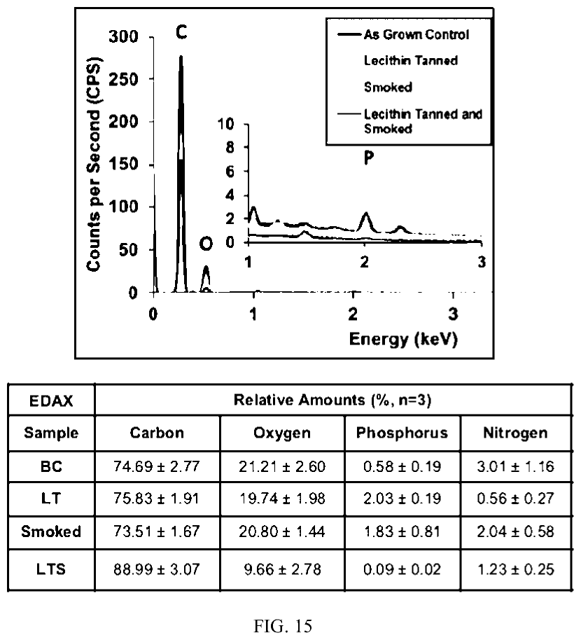

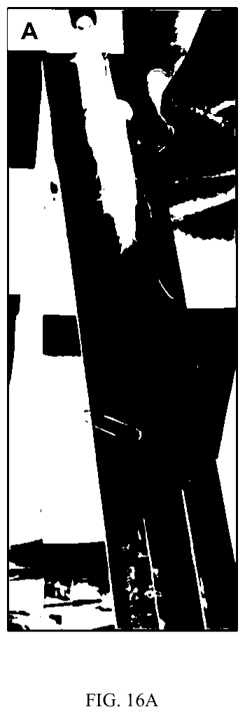

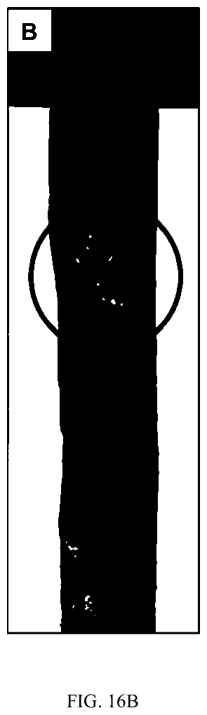

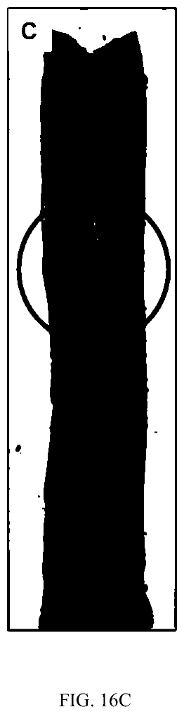

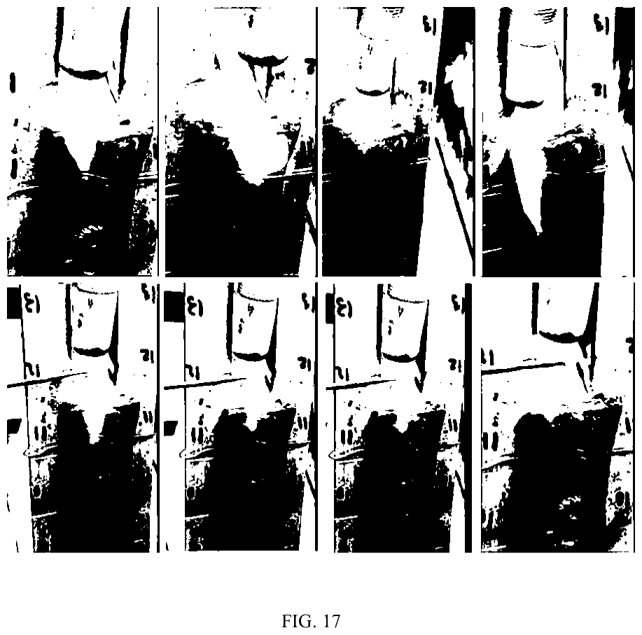

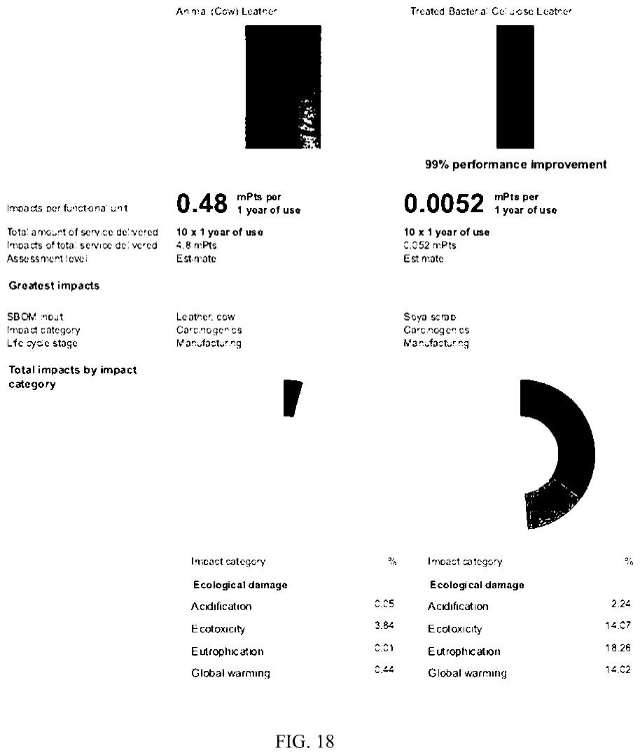

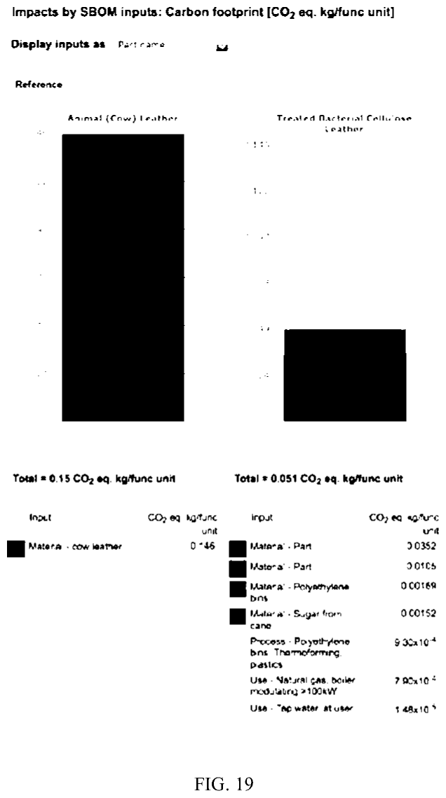

### Methods for Results Shown in FIGS. 1-19

### Scanning Electron Microscopy (SEM)

Hydrated microbial cellulose samples were initially placed in a −20° C. freezer for 24 hours and lyophilized in a freeze dryer system (Labconco FreeZone, Kansas City, Mo.) for 24 hours at −84° C. and 2.0×10⁻² mbar. Microbial cellulose morphology was assessed by using a scanning electron microscope (Zeiss Sigma VP, Oberkochen, Germany; 3 kV; n=5). Prior to imaging, samples were sputter coated with 30 nm of gold. The fiber diameter of each sample was measured by analyzing 100 randomly selected fiber segments in SEM images using NIH lmageJ software, in which their diameters were measured to calculate an average fiber diameter.

### Energy-Dispersive X-Ray Spectroscopy (EDS)

Microbial cellulose surface elemental characterization was assessed by EDS (10 kV; 5 minutes; n=3). Prior to analysis, samples were sputter coated with Cu.

### Thermogravimetric Analysis (TGA)

Thermal properties of dried, pre-weighed microbial cellulose discs (Diameter: 4.5 mm) were determined by a thermogravimetric analyzer (TGA 550, TA Instruments). TGA analysis was performed at a heating rate of 10° C./min over the temperature range of 25° C. to 700° C. under flowing nitrogen (40 ml/min).

### Fourier Transform Infrared Spectroscopy (FTIR)

Chemical conformational characteristics of microbial cellulose biofilms were assessed using Fourier transform infrared (FTIR) spectroscopy (LUMOS, Bruker). Spectra were collected in attenuated total reflectance (ATR) mode from 600-4000 cm⁻¹ using 200 scans with a resolution of 4 cm. For each biofilm, three samples of the same conditions were examined.

### Swell Test

Dried, pre-weighed microbial cellulose discs (Diameter: 4.5 mm, n=5) were swollen in 7 ml of deionized water at room temperature for 24 hours. The degree of swelling of the microbial cellulose were measured after 20 min, 1 h, 2 h, 4 h, and 24 h. The degree of swelling was calculated as the following:

Degree of swelling=[(Wet weight−Dry weight)/Dry weight]×100%

### Mechanical Testing

For tensile testing, the microbial cellulose samples (n=3) were secured with custom clamps and mounted in an Instron (Model 11321, Norwood, Mass.), equipped with a 25 kN load cell. Samples were maintained to have a gauge length of 2 in. and were tested to failure. Microbial cellulose elastic modulus, yield strength, ultimate tensile strength, toughness, and resilience were determined from the stress-strain curve.

### X-ray Photoelectron Spectroscopy (XPS)

XPS the BC films specimens was obtained with a PHI 5500 XPS and electron analyzer. The spectra were recorded using a monochromatic Mg-Ka radiation X-ray source (hv=1253.6 eV) and the analyzer pass energy was set to 25 eV., with 50 W operating at 15 kV voltage and a base pressure of 2×10⁸ ton in the sample chamber. The XPS spectra were collected in the range, 0-1200 eV, with a resolution of 0.1-1.0 eV. The inelastic background of the C1s, O1s, N1s and P 2p electron core spectra was subtracted using Shirley's method and data was analyzed using commercial, curve fitting software. The binding energy scale was calibrated with reference to the C1s line at 285.0 eV.

### Absorbency of Textiles (AATCC TM79)

Time it takes for a droplet of water to fully absorb into a textile material.

### 45° Flame Test ASTM D1230-94

Standard Test Method for Flammability of Apparel Textiles. This test is used to measure and describe the properties of natural or synthetic fabrics in response to heat and flame under controlled lab conditions. Most any textile material can be evaluated using this test. Two factors are measured: 1) Ease of ignition (how fast the sample catches on fire). 2. Flame spread time (the time it takes for the flame to spread a certain distance). Samples are mounted in a frame and held in a special apparatus at an angle of 45°. A standardized flame is applied to the surface near the lower end for specified amount of time. The flame travels up the length of the fabric to a trigger string, which drops a weight to stop the timer when burned through. The time for the flame to travel the length of the fabric and break the trigger string is recorded, as well as the fabric. Scientific Testing Requirements: Condition according to ASTM D1776-98.

### Experimental Set 2

### Microbial Biotextiles for a Circular Economy

### Overview

The data below demonstrates utilization of microbial biosynthesis and adaptation of ancient textile techniques for the sustainable development of regenerative, high performance biotextiles, highlighting how biofabrication and green processing can strategically address the most damaging impacts of a linear economy, as encapsulated by the fashion industry.

The linear economy that has been the dominant production model since the Industrial Revolution presents serious ecological and human health concerns and potentially catastrophic climate instability. In particular, the textile industry-reliant on industrial agriculture for cellulosic fibers (M. A. Altieri, Ecological Impacts of Industrial Agriculture and the Possibilities for Truly Sustainable Farming. Monthly Review 50, 3 (1998)), and nonrenewable resources and petrochemicals to produce synthetic fibers, dyes, tanning and finishing agents, as well as chemically and energy intensive processing (S. S. Muthu, “Assessing the Environmental Impact of Textiles and the Clothing Supply Chain” (Woodhead Publishing, 2nd Edition. 2020) is currently responsible for 10% of global carbon emissions (P. Chrobot, M. Faist, L. Gustavus, A. Martin, A. Stamm, R. Zah, M. Zollinger, “Measuring Fashion: Insights from the Environmental Impact of the Global Apparel and Footwear Industries study.” (Quantis, 2018)), 20% of global waste water (World Bank “The Bangladesh Responsible Sourcing Initiative: A New Model for Green Growth?” (World Bank, 2014)), 35% of marine microplastic pollution (A. Hulse, “Engineering Out of Fashion Waste” (Institution of Mechanical Engineers, 2018)), and expected to use 25% of the global carbon budget by 2050 (A New Textiles Economy: Redesigning Fashion's Future. Ellen MacCarthur Foundation Report, 28 Nov. 2017, p. 21). Microplastics, which contain endocrine disrupting chemicals and readily absorb and accumulate persistent organic pollutants, have been found in the gastrointestinal tract of marine animals, the intestines of humans, and, recently, in human placentas, where they may trigger immune response and release toxic chemicals (EFSA Panel on Contaminants in the Food Chain (CONTAM), EFSA J. 14, 4501-4531, (2016); Y. Deng, Y. Zhang, B. Lemos, H. Ren, Tissue accumulation of microplastics in mice and biomarker responses suggest widespread health risks of exposure. Sci Rep 7, 46687 (2017); J. J. Reineke, D. Y. Cho, Y.-T. Dingle, A. P. Morello, J. Jacob, C. G. Thanos, E. Mathiowitz. Unique insights into the intestinal absorption, transit, and subsequent biodistribution of polymer-derived microspheres. Proc. Natl. Acad. Sci. 110, 13803-13808 (2013); A. Ragusa, A. Svelato, C. Santacroce, P. Catalano, V. Notarstefano, O. Carnevali, F. Papa, M. C. A. Rongioletti, F. Baiocco, S. Draghi, E. D'Amore, D. Rinaldo, M. Matta, E. Giorgini, Plasticenta: First evidence of microplastics in human placenta. Environ. Int. 146, 106274 (2021); S. L. Wright, F. J. Kelly, Plastic and Human Health: A Micro Issue? Environ. Sci. Technol. 51, 6634-6647 (2017)). Thus, there is a pressing need for new fabrication strategies to design functional biomaterials and green chemistry processes that facilitate a transition to a sustainable, circular economy.

Biofabrication has emerged as a strategy to produce biologically functional products with structural organization from living cells (eukaryotic or prokaryotic), hybrid tissue constructs, and/or biomaterials either through top-down (bioprinting) or bottom-up (bio-assembly) approaches. Motivated by a ‘green’ bio-economy, various biofabrication technologies (3D printing, green electrospinning, microbial fermentation) provide significant opportunities for developing regenerative, non-toxic biomaterials with a closed loop life cycle. The climate change mitigation potential of biofabrication processes and products is estimated at 1-2.5 billion tons CO₂ equivalent per year by 2030; more than the total reported emissions for Germany in 1990|_{[TS1]} (Industrial Biotechnology and Climate Change Organisation for Economic Co-operation and Development (OECD) 2011 report. oecd.org/sti/emerging-tech/49024032.pdf); and bioplastics alone have the potential to mitigate 3.8 Gigatonnes of CO₂ by 2050 (P. Hawken, Ed. Drawdown: The Most Comprehensive Plan Ever Proposed to Reverse Global Warming. Penguin Books, 2017).

Microbial cellulose (MC) is a highly crystalline and chemically pure biopolymer, free of hemicellulose and lignin, produced extracellularly by gram-negative bacteria, such as Gluconacetobacter xylinus (G. xylinus), formerly known as Acetobacter Xylinum (C. Plieth, Calcium: Just Another Regulator in the Machinery of Life? Annals of Botany 96, 9-21 (2005); H. Shibazaki, S. Kuga, F. Onabe, M. Usuda, Bacterial cellulose membrane as separation medium. J Appl. Polym. Sci. 50, 965-969 (1993); A. F. Jozala, L. C. de Lencastre-Novaes, A. M. Lopes, V. de Carvalho Santos-Ebinuma, P. G. Mazzola, A. Pessoa Jr, D. Grotto, M. Gerenutti, M. V. Chaud, Bacterial nanocellulose production and application: a 10-year overview. Appl. Microbiol. Biotechnol. 100, 2063-72 (2016)). Under specific aerobic culturing conditions these bacteria biosynthesize unaligned cellulose nanofibrils (10-100 nm diameter) that coagulate into a three-dimensional layered hydrogel (>98% water), at the air-culture media interface, commonly referred to as a pellicle. Despite having the same chemical structure as plant-based cellulose, MC is uniquely characterized by a natural self-assembled nanofiber network, high swelling capability, moldability, and robust tensile strength as a result of high degrees of polymerization and crystallinity (80-90%) (M. Gama, P. Gatenholmama, D. Klemm, Ed., Bacterial NanoCellulose: A Sophisticated Multifunctional Material (CRC Press, 2012); S. P. Lin, I. Loira Calvar, J. M. Catchmark, J. R. Liu, A. Demirci, K. C. Cheng, Biosynthesis, Production and Applications of Bacterial Cellulose. Cellulose 20, 2191-2219 (2013); K. Y. Lee, G. Buldum, A. Mantalaris, A. Bismarck, More than Meets the Eye in Bacterial Cellulose: Biosynthesis, Bioprocessing, and Applications in Advanced Fiber Composites. Macromol Biosci 14, 10-32 (2014); B. V. Mohite, S. V. Patil, A Novel, Biomaterial: Bacterial Cellulose and its New Era Applications. Biotech. Appl. Bioc. 61, 101-110 (2014)). One of the most intriguing aspects of MC is its tunability of material properties and functionality during biosynthesis and green chemistry processing, which make it a highly promising modular engineering platform for advanced, regenerative, biomimetic materials in a wide range of fields, from textiles (A. F. De Santana Costa, A. M. Vasconcelos Rocha, L. A. Sarubbo, Review—Bacterial Cellulose: An Ecofriendly Biotextile. IJTFT 7, 11-26 (2017)) to healthcare (S. Gorgieva, J. Trěcek, Bacterial Cellulose: Production, Modification and Perspectives in Biomedical Applications. Nanomaterials 9, 1352 (2019); J. Li, Y. Wan, L. Li, H. Liang, J. Wang, Preparation and Characterization of 2,3-dialdehyde Bacterial Cellulose for Potential Biodegradable Tissue Engineering Scaffolds. Mater. Sci. Eng. C. 29, 1635-1642 (2009)) and electronics (T. Bayer, B. V. Cunning, R. Selyanchyn, M. Nishihara, S. Fujikawa, K, Sasaki, S. M. Lyth,

High Temperature Proton Conduction in Nanocellulose Membranes: Paper Fuel Cells. Chem. Mater. 28, 4805-4814 (2016)).

MC's bottom-up approach and material properties offer distinct advantages for textiles that enable low impact processing and minimal waste production: MC grows to the shape of the cultivation vessel with no purification required; and physical properties (tensile strength, crystallinity, hydrophilicity, porosity) can be controlled during biosynthesis and with green processing. Donini et. al. reported that the amount of cellulose produced by eucalyptus in a 10,000 m² area of land over 7 years could be achieved at higher purity by microbial fermentation in a 500 m³ bioreactor in only 22 days (Í. Donini, D. De Salvi, F. Fukumoto, W. Lustri, H. Barud, R. Marchetto, Y. Messaddeq, S. Ribeiro, Biosynthesis and Recent Advances in Production of Bacterial Cellulose. Ecl. Quim. 35, 165-178 (2010)). As such, microbial nanocellulose can offer a rapidly renewable, high purity raw material alternative for regenerative, performance biotextiles at industrial scale while eliminating the land, water and chemicals usage of production of plant-based cellulose (G. Chen, G. Wu, L. Chen, W. Wang, F F. Hong, L J. Jönsson, Comparison of Productivity and Quality of Bacterial Nanocellulose Synthesized Using Culture Media Based on Seven Sugars from Biomass. Microb. Biotechnol. 12, 677-687 (2019)) and nanocellulose production from wood pulp (Q. Li, S. McGinnis, C. Sydnor, A. Wong, S. Renneckar, Nanocellulose Life Cycle Assessment. ACS Sustainable Chem. Eng. 1, 919-928 (2013)), with superior tensile strength. In addition, the ability of G. xylinus to produce MC from a variety of carbon and nitrogen sources enables the opportunity to extract fermentation nutrients from agro-industrial by-products and waste streams; opening pathways for biofabrication of materials with both environmental and economic benefit at industrial scale (V. Revin, E. Liyaskina, M. Nazarkina, A. Bogatyreva, M. Shchankin, Cost-Effective Production of Bacterial Cellulose Using Acidic Food Industry By-Products. Braz. J. Microb. 49, 151-159 (2018)).

MC has great potential to meet key design criteria—renewability, scalability, low toxicity, tunability, compostability and performance—for multi-functional, adaptable, regenerative textiles. When grown to sufficient thickness (>2 cm), MC biofilms can be dehydrated to create a leather-like material as the nanofiber network collapses into a dense mesh. However, like many naturally occurring biopolymers for which water acts as a natural plasticizer (M. G. A. Vieira, M. A. da Silva, L. O. dos Santos, M. M. Beppu, Natural-Based Plasticizers and Biopolymer Films: A Review, Eur. Polym. J. 47, 254-263 (2011)), the high hygroscopicity of unmodified MC results in a brittleness during thermoformation and variability in mechanical properties that limit translation to textile applications. For instance, when untreated, microbial cellulose and animal hides dehydrate to a biomaterial with the properties of parchment, or raw hide, depending on the initial thickness of the material in the hydrated state and drying conditions. Strategies to regulate the hydration state and subsequent mechanical properties of biopolymers to meet design criteria for textile applications, including tensile strength and flexibility, often involve heavy metals and synthetic plasticizers, which compromise material biodegradability and introduce toxicity.

For instance, current leather production is dominated by chrome-tanning, creating large volumes of highly toxic, mutagenic, teratogenic, and carcinogenic chemical pollution, such that tannery effluents are ranked as the highest pollutants among all industrial wastes (A. G. Khan. Relationships between chromium biomagnification ratio, accumulation factor, and mycorrhizae in plants growing on tannery effluent-polluted soil. Environ, Int. 26, 5-6, 417-423 (2001); G. L. Tadesse, T. K. Guya, Impacts of Tannery Effluent on Environments and Human Health. Environ. 7, 3 (2017); M. M. Altaf, F. Masood, A. Malik, Impact of Long-Term Application of Treated Tannery Effluents on the Emergence of Resistance Traits in Rhizobium sp. Isolated from Trifolium Alexandrinum. Turk. J. Biol. 32, 1-8 (2008)). In India alone, about 2000-3000 tons of chromium escapes into the environment annually from tannery industries, contaminating all nearby surface and groundwater systems (M. A. H. Bhuiyan, N. I. Suruvi, S. B. Dampare, M. A. Islam, S. B. Quraishi, S. Ganyaglo, S. Suzuki, Investigation of the Possible Sources of Heavy Metal Contamination in Lagoon and Canal Water in the Tannery industrial Area in Dhaka, Bangladesh. Environ. Monit. Assess. 175, 633-649 (2011)) with aqueous effluent chromium concentrations ranging between 2000 and 5000 mg/l, seriously exceeding the permissible discharge limits of 2 mg/l (D. Saranya, S. Shanthakumar, Opportunities for phycoremediation approach in tannery effluent: A treatment perspective. Environ. Prog. Sustain. 38, 3, 1-13 (2019)).

Combining microbial biofabrication with observations from ancient textile techniques and indigenous science (J. T. Johnson, R. Howitt, G. Cajete, F. Berkes, R. P. Louis, A. Kliskey, Weaving Indigenous and Sustainability Sciences to Diversify our Methods. Sustain Sci. 11, 1-11 (2016)) can inform the development of sustainably engineered, performance biotextiles. For millennia, societies around the world have extracted natural color from plants, insects and minerals (E. S. B. Ferreira, A. N. Hulme, H. McNab, A. Quye, The Natural Constituents of Historical Textile Dyes. Chem. Soc. Rev. 33, 329-336 (2004)), and used a variety of non-toxic tanning methods, including brain and organ (“fat”) tanning followed by smoking (aldehyde tanning), to create durable, water-repellent leather textiles dating back at least 5,000 years to “Ötzi” (“Iceman”) (A. G. Püntenera, S. Moss, Ötzi, the Iceman and his Leather Clothes. Chimia 64, 315-320 (2010)). Brain tissue is high in fat and oil content, as well as the phospholipid lecithin, which serves to lubricate and soften the fibrous structure of the hides and increase flexibility. While the detailed chemistry of brain tanning is not fully understood, it is, like smoking, expected to be a form of aldehyde tanning (C. L. Heth, “The Skin They Were In: Leather and Tanning in Antiquity” in Chemical Technology in Antiquity, ACS Symposium Series 1211, 181-196 (2015)). [TS2]The lipid portion of the lecithin molecules polarize away from water, phosphate group of lecithin is attracted to the water solution, which stabilizes the tanning emulsion and likely facilitates the interaction of the oils with the collagen in leather.

Here, inspired by the complexity of nature and its robust regenerative potential, the inventors harness microbial biosynthesis and adapt ancient textile techniques for the development of regenerative, high performance, sustainable biotextiles. In particular, microbial cellulose is produced from G. xylinus and treated with a phospholipid emulsion of sunflower seed oil and lecithin phosphatidylcholine that alters the chemical, thermal, and mechanical properties of the material to meet design criteria for sustainable textile applications. The lecithin treatment increases the tensile strength and flexibility of MC, and imbues the biotextile with extreme flame retardance. Life cycle impact assessment (LCA) shows that the biofabrication method for MC biotextiles offers a dramatic reduction in environmental damage relative to manufacture of conventional textiles, including leather, synthetic leather, and cotton. Our findings highlight the potential of microbial biosynthesis to facilitate a transition to a circular economy, in which rapidly renewable resources, waste streams, and low impact processes are used to fabricate regenerative, high performance biomaterials characterized by renewability, scalability, biocompatibility (G. Helenius, H. Bäckdahl, A. Bodin, U. Nannmark, P. Gatenholm, B. Risberg, In Vivo Biocompatibility of Bacterial Cellulose. J. Biomed Mater Res. A. 76, 431-8 (2006)), and biodegradability.

### Results and Discussion

### Biomaterial Synthesis and Processing in a Circular Life Cycle

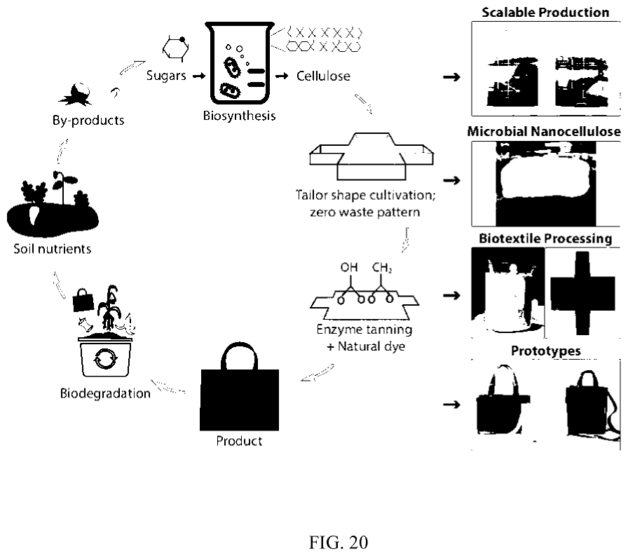

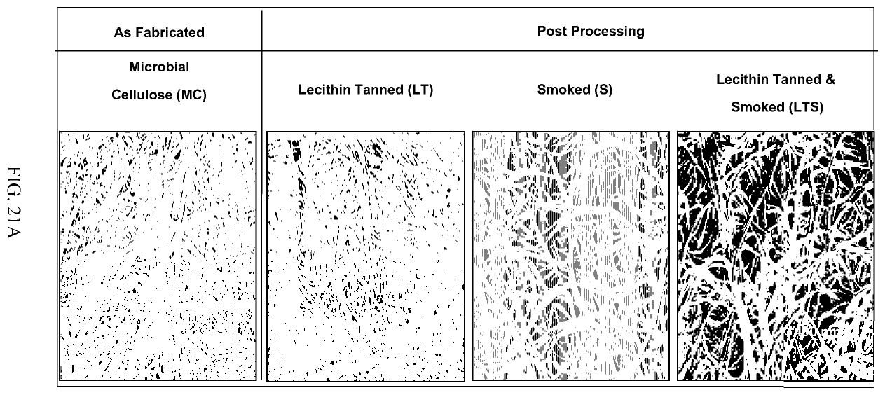

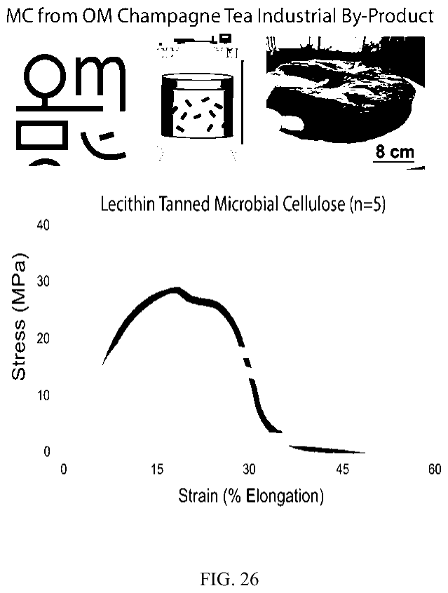

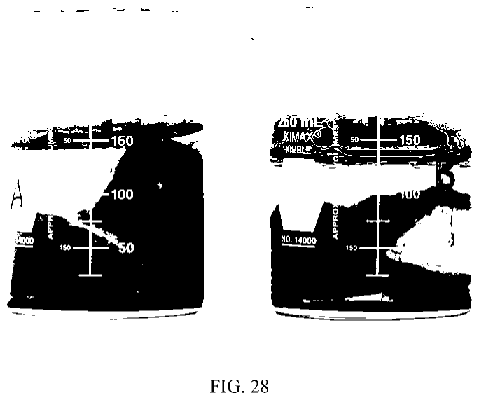

FIG. 20 illustrates a schematic of our bottom-up approach that utilizes microbial biosynthesis and processing based in green chemistry to create high regenerative, high performance products with a closed loop life cycle. MC textiles are biofabricated from bacteria, such as G. xylinus, that metabolize carbon sources into a nanocellulose fibrous network via enzymatic pathways. Scalable production from a variety of carbon sources, such as glucose, fructose, and sucrose, which may be derived from agro-industrial byproducts, highlights this platform's vast utilization of renewable resources (FIG. 26); FIG. 28 shows a frame from a time lapse of microbial cellulose biosynthesis using different carbon sources (see https://vimeo.com/492333189). Due to biosynthesis of the biofilm at the air-media interface, stabilization of desired engineered morphologies and geometries can be controlled as cellulose self-assembles into the shape of the growth vessel, enabling zero waste patterning. Treatment of microbial cellulose textiles with a sunflower seed-based lecithin emulsion and natural dyes serve as non-toxic processing techniques to create high performance textile products, such as our bag prototypes shown in FIG. 20. MC-based products are degraded once exposed to environments rich with cellulolytic microorganisms (terrestrial and aquatic) that hydrolyze the cellulose's linear β-1,4-glycosidic linkages, so they may reenter the ecosystem as nutrients at the end of their useful life. MC is reported to be non-cytotoxic and non-cytogenetic (D. Klemm, F. Kramer, S, Moritz, T. Lindstrom, M. Ankerfors, D. Gray, A. Dorris, Nanocelluloses: A New Family of Nature-Based Materials Angew. Chem. Int. Ed. 50, 24, 5438-5466 (2011)). Inspired by the ability to adapt ancient textile techniques for leather to modify cellulose's cross-linked network and potential to satisfy design criteria for sustainable textile applications, the inventors also investigated following the lecithin tanning (LT) process with an aldehyde smoke treatment (S). Textiles prepared from untreated, lecithin tanned, aldehyde tanned (smoked), and lecithin and aldehyde tanned are denoted as MC, LT, S, and LTS, respectively (see below Methods section for details).

### Characterization

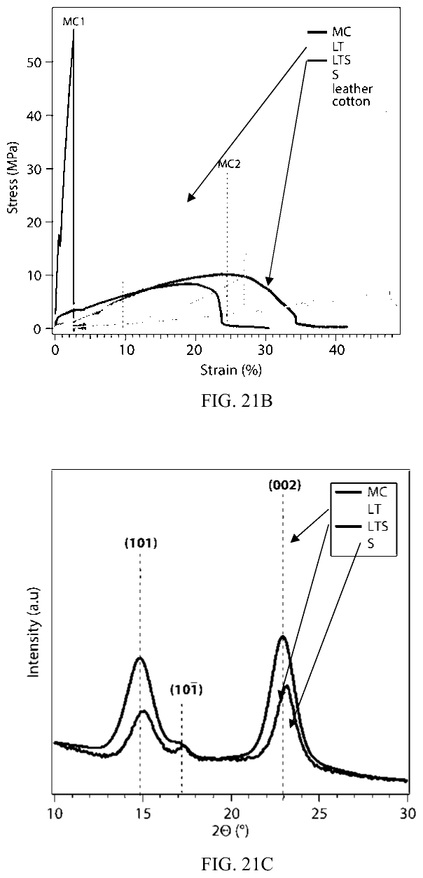

Scanning electron microscopy (SEM) images shown in FIG. 21A reveal a dense three-dimensional nanofiber network on all microbial cellulose surfaces before and after treatments. The unaligned and intertwined nanofibrous structures are connected by the natural polymer's extensive inter- and intramolecular hydrogen bonding. Both pristine and treated microbial cellulose exhibited similar well-organized three-dimensional unaligned fibrous networks. Absence of isolated lecithin spheres on the surface of nanofibers after immersion indicate that minimal amounts of the phospholipid immobilized to cellulose, corresponding to lecithin's weak physical adsorption (J. Zhang, P. Chang, C. Zhang, G. Xiong, H. Luo, Y. Zhu, K. Ren, F. Yao, Y. Wan, Immobilization of lecithin on bacterial cellulose nanofibers for improved biological functions. React. Funct. Polym. 91, 100-107 (2015)). In comparison to traditional cotton fibers with a diameter of 12.70±3.11 μm, as-fabricated microbial cellulose fibers have an average diameter of 71.00±19.00 nm (n=100; Table 1). Treatment with lecithin and smoke tanning, separately and sequentially, exhibited no significant differences in fiber size between groups. This indicates that the lecithin emulsion is not coating the fibers, which would correspond to an increased diameter, but rather have a chemical effect on the MC matrix.

Uni-axial tensile mechanical analysis demonstrated that pristine microbial cellulose bio-textiles have varied stress-strain profiles that can have elastic moduli between 58.30±35.71 MPa (MC I) and 210.91±58.61 MPa (MC II) shown in Table 1; representative spectra for MC 1 (high modulus, brittle) and MC (lower modulus, greater elongation at break) is shown in FIG. 21B. Without lecithin treatment, smoked MC (S) showed similar variability in elongation at break, but a reduced modulus and tensile strength. Treatment with lecithin emulsion (LT) significantly increased the elastic modulus, toughness, and maximum stress (\hat{ } p<0.05, n=4), and stabilized the mechanics to create a robust biotextile with greater tensile strength than leather or cotton up to strain elongation values of 33% and 45%, respectively (FIG. 21B). Following the lecithin treatment with aldehyde smoke treatment (S) leads to a reduction in tensile strength and toughness.

Table 1 displays the water contact angle (WCA) for microbial cellulose samples before and after treatments. While lecithin is a natural amphiphilic phospholipid composed of a charged hydrophilic head group and a hydrophobic hydrocarbon tail, smoke tanning exposes samples to a hydrocarbon-rich environment. This modulation of microbial cellulose hydrophilicity facilitated by minimal phospholipid immobilization and aldehyde groups indicates that these treatments chemically modify the cellulose hydrogen bonded network. The lecithin treatment can be followed by an aldehyde treatment to add water resistance to the MC biotextile. However, the breaking of unsaturated bonds in fatty acids during their degradation over time is also a source of aldehydes in low concentration, which help give the MC water resistance due to the hydrophobic nature of the fats, and provide a source for aldehyde tanning A. G. Püntenera, S. Moss, Ötzi, the Iceman and his Leather Clothes. Chimia 64, 315-320 (2010).

XRD data shown in FIG. 21C indicates that lecithin treatment shifts the MC peaks towards lower 20, corresponding to an increase in d-spacing relative to as fabricated MC of 1.22% and 0.82% for the [101] and [002] reflections, respectively, as calculated with Bragg's equation. An increase in crystallinity index (CI) calculated using Segal's method accompanies the increase in lattice spacing for LT MC (Table 1). The higher CI is consistent with the increase in tensile strength of LT MC, while the larger d-spacing is expected to relate to the increased flexibility, providing insights for the unusual increase in both tensile strength and elastic modulus, usually trade-offs, observed after lecithin treatment. After vacuum anneal at 85° C., a decrease in crystallinity and lattice spacing is observed for LT MC, suggesting that hydration state may influence the mechanics. Water molecules reduce the glass transition temperature of biopolymers, improving flexibility and processability, as such are considered natural plasticizers (Q. Li, S. McGinnis, C. Sydnor, A. Wong, S. Renneckar, Nanocellulose Life Cycle Assessment. ACS Sustainable Chem. Eng. 1, 919-928 (2013)). Likewise, lecithin offers hydrophobic and hydrophilic sites to increase interaction between cellulose and oils, which may modify the cellulose cross-linking so as to be effective as a low toxicity, non-migratory plasticizer (M. Pommet, A. Redl, M. Morel, S. Guilbert, Study of Wheat Gluten Plasticization with Fatty Acids. Polymer 44, 115-122 (2003)). Consistent with SEM images (FIG. 21A), no lecithin peaks appear in the data. Accordingly, the increased tensile strength and flexibility observed for LT MC may be attributed to an increased hydration state of the material after treatment and/or change in cross-linking, which can explain the enhanced stability and mechanical properties of LT MC relative to the untreated biomaterial.

### Chemical Analysis

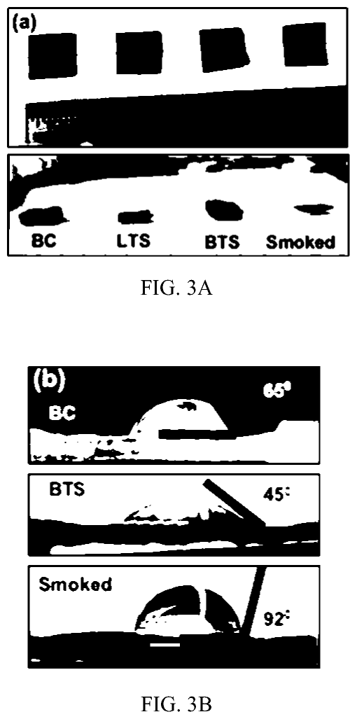

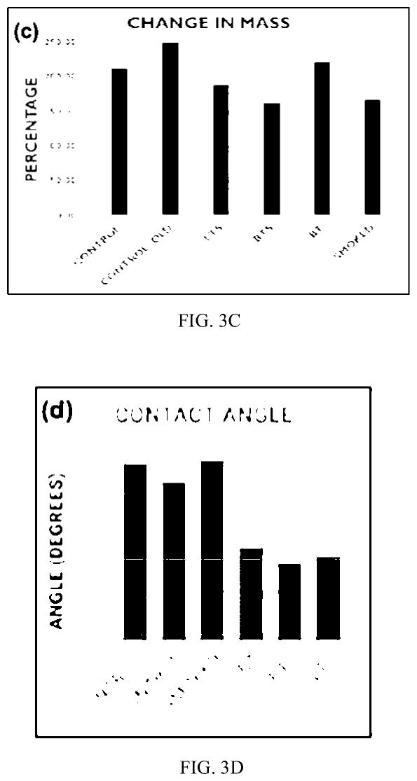

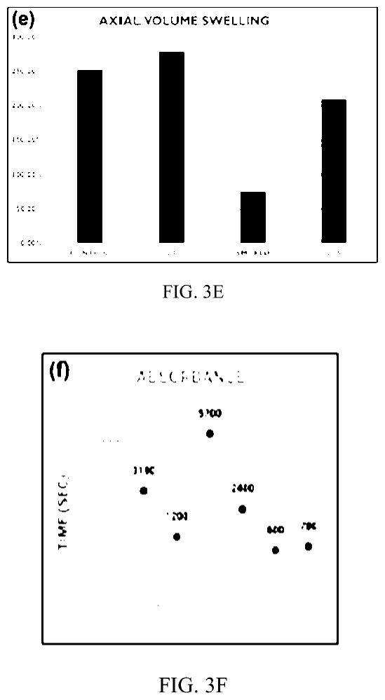

C 1s XPS data shown in FIG. 3a after 24 hour vacuum anneal at 75 C shows at the lecithin treatment is accompanied by an increase in surface C—O—C and C—H bonding, as well as —COOR groups (carboxylic acids, esters and anhydrides). The broadening and change in line shape of the O 1s core level peak for lecithin tanned (LT) MC is attributed to an increase in C—O, O—C—O, and —COOR groups at the surface of the treated sample. The P 2p data shows phosphorus is introduced to the material by the tanning treatment in oxidized form.

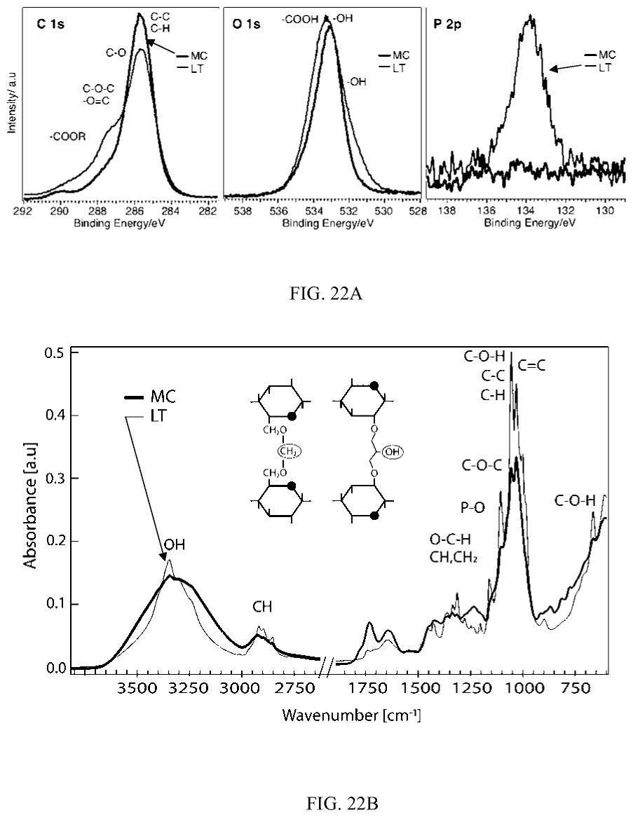

FTIR data in FIG. 22A shows the introduction of P—O vibrations at 1207.37 cm⁻¹ with lecithin tanning (R. Nirmala, et al. Mater. Sci. Eng., C, 31 (2011), pp. 486-493; E. V. Shumilina Mater. Sci. Eng. C, 3 (1995), pp. 43-50). However, lecithin absorption peaks are not observed, consistent with the SEM micrographs (FIG. 21A), showing the fibers are not coated with lecithin. The overall sharpening and increase in intensity of the FTIR peaks for LT compared to as fabricated MC, suggests that lecithin tanning results in a more well-defined bond environment as well as introduces chemical groups which modify the cross-linking and subsequently increase the tensile strength and flexibility of MC. In particular, the increase and sharpening of the band between 3450 cm⁻¹ and 3000 cm⁻¹ indicates that the increase in hydration state due to lecithin tanning allows hydroxyl groups to form a well-defined bonding environment. This is consistent with the stability of the higher hydration state indicated by XRD data (FIG. 21C) and is expected to result in increased reactivity of cellulose through hydroxyl groups available for bonding. In addition, there is a clear sharpening of intensity increase in absorption bands related to CH, CH₂, and C—O—H groups appearing in the regions 3000-2853 cm⁻¹, 1437-1245 cm⁻¹, 1184-1104 cm⁻¹, and at 690 cm⁻¹ The series of sharp peaks between 2853 cm⁻¹ and 3000.6 cm⁻¹ are assigned to the carbon-hydrogen (CH) band, while asymmetric and symmetric C—H stretching vibrations of methyl and methylene groups appear at 2850 to 2950 cm⁻¹ (N. Zhu, F. Z. Cui, K. Hu, L. Zhu J. Biomed. Mater. Res., 82A (2007), pp. 455-461; Y. Wan, G. Zuo, C. Liu, X. Li, F. He, K. Ren, H. Luo Polym. Adv. Technol., 22 (2011), pp. 2659-2664). This band is broad and fairly weak for as grown MC, but sharpens and increases in intensity for LT MC, and a weak but sharp C—H stretch appears at 3000.6 cm⁻¹ which is not observed for as grown MC. This suggests that the exocyclic CH₂ group may be involved in physical or chemical interactions during the treatments. Changes are also observed at 1428, 1370 and 1316 cm⁻¹, which are related to the C—H deformation. Further FTIR analysis provides insights into the relative amount of crystallinity in cellulosic materials, also known as the crystallinity index. The crystallinity index is measured by the Lateral Order Index (LOI, A1420/A893), in which the absorbance at 1420 and 893 cm-1 are indicative of the amount of crystalline and amorphous regions, respectively (M. Fan, D. Dai, B. Huang, Fourier transform infrared spectroscopy for natural fibres in Fourier transform-materials analysis Dr Salih Salih (Ed.) 2012, 3, pp. 45-68; H. Guo, M. He, R. Huang, W. Qi, W. Guo, R. Su, Z. He, Changes in the supramolecular structures of cellulose after hydrolysis studied by terahertz spectroscopy and other methods. RSC Adv 4 101, 57945-57952 (2014)). An increase in LOI in lecithin tanned MC compared to as fabricated samples, demonstrated that this treatment created a more ordered structure with a higher degree of crystallinity.

Collectively, the data suggests that the lecithin tanning processes introduces sites for chemical cross-linking through methylene and hydroxyl groups, shown schematically in the FIG. 22A inset. Methylene groups may form upon reaction of new chemical species with hydroxyl group at the 6 position, which, bonded to only one alkyl group in the anhydroglucose unit, acts as a primary alcohol and can react ten times faster than the other OH groups (A. Hebeish, J. T. Guthrie, The Chemistry and Technology of Cellulose Copolymers, No. 4 in Polymers—Properties and Applications. Springer-Verlag, 1981), while intermolecular bonding through carboxyl groups may proceed by attachment at the hydroxyl groups at the 6 position, found to have twice the reactivity of that at the 3 position (A. Dufrense, Chemical Modification of Nanocellulose in Nanocellulose: From Nature to High Performance Tailored Materials. Berlin: De Gruyter, 2012, pp. 147-149).

### Flame and Thermal Properties

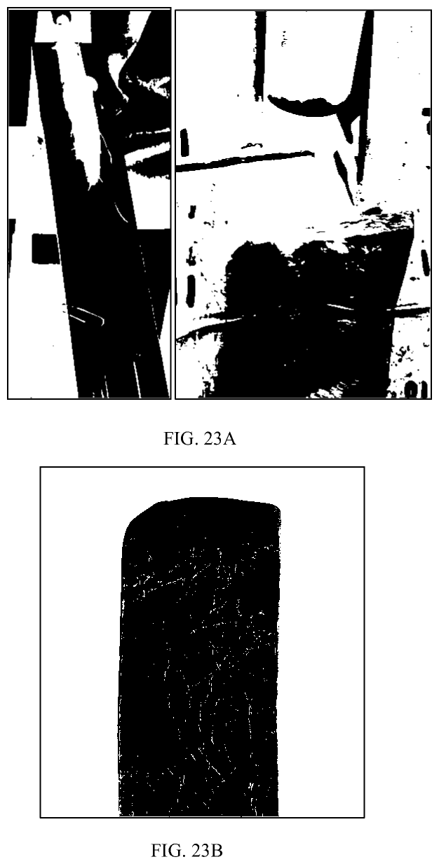

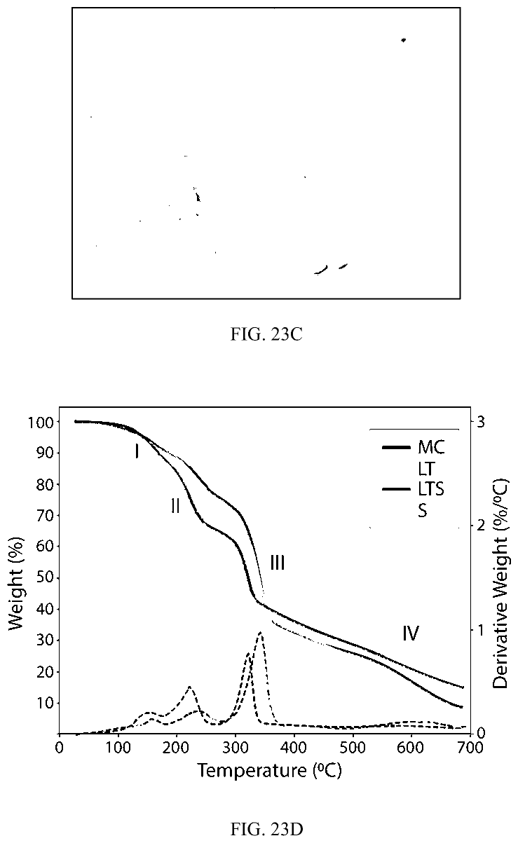

Since flame retardants containing phosphorus are increasingly successful as halogen-free alternatives for polymeric materials, the inventors investigated the flammability of as fabricated and lecithin and aldehyde treated microbial cellulose. Flammability was determined using a 45° flame test (ASTM D1230-94), which utilizes controlled lab conditions to describe the ease of ignition and spread of fire on natural or synthetic fabrics. FIG. 23A displays images of LT microbial cellulose during its exposure to a direct 2054° F. flame. Upon application of the flame, microbial cellulose samples did not ignite, and the entirety of the biotextile was preserved. The control aluminum rod melted during the test, while the biotextile deflected the flame, thus avoiding flame propagation. Following, LT MC was repeatedly exposed to the direct flame until the cellulose samples charred on their surface. Despite the repeated exposure and surface charring, the bulk biomaterial was maintained, further signifying the presence of significant flame-retardant capacity (FIG. 23B). Untreated MC also demonstrated flame retardance, but were observed to char more readily than LT samples which deflected the flame at the sample surface (FIG. 23A, right). Inconsistent with the ignitable behavior of typical cotton-based cellulose and synthetic fibers, microbial cellulose's physical, morphological, and chemical properties are known to reduce heat transmission from the flame to the biomaterial and volatile release towards the flame. For both treated and untreated MC, the degree of flame retardance increases with the thickness of the MC pellicle, and consequently the number of layers biosynthesized via layer by layer biofilm growth at the air-media interface.

In high magnification SEM images of the charred pristine microbial cellulose biotextiles it can be observed that intumescent bubbles are formed on the fiber surface as a result of the reaction to the fire (FIG. 23C). Energy dispersive x-ray (EDX) analysis indicated the presence of carbon, oxygen, and significantly increased amounts of phosphorus on lecithin treated microbial cellulose surfaces. The formation of intumescent bubbles is the result of the phosphoric acid production at high temperatures, causing the cellulose polymer to dehydrate and the generation water vapor, favoring the production of foaming char. An insulating char barrier prevents combustion via the reduction of heat transmission, and diffusion of oxygen and combustible volatiles. Additionally, this multi-layer structure mimics a multilayer nanocoating that are well known as physical surface barriers that limit heat and oxygen transfer between the fire and cellulose.

The thermal stability and decomposition behavior of microbial cellulose before and after lecithin or aldehyde (smoking) treatment was determined by thermogravimetric analysis (TGA) under a nitrogen atmosphere from 25-680° C. (FIG. 23D, n=5). The TGA and dTGA curves of untreated microbial cellulose show a three-step weight loss between 25-200° C., 210-240° C., and 300-360° C., representing the initial evaporation of free and bound water (I), followed by polymer decomposition (II-III), and the production of either polymer pyrolysis-based levoglucosan or flame-resistant char (IV). Accordingly, region I provides insights into the hydration state, while the peak positions in region II (210-240° C.) and intensity in region III and IV are related to chemical pathways of thermal decomposition.

In region I, microbial cellulose's maximum weight loss rate is 5.38±0.87% at 150° C. (Table 2). Throughout the second decomposition phase, and the third decomposition phase (300-360° C.), TGA curves shows a maximum weight loss rate of 16.96±2.37% at 232° C., and 49.51±1.21% at 340° C. (Tₘₐₓ), respectively. Following, the aliphatic compounds are decomposed into char with a residual mass of 8.99% at 680° C. Lecithin treated microbial cellulose biotextiles show an increased initial mass loss of 8.50±0.25% at 150° C. with respect to as-fabricated cellulose, indicating a higher hydration state, consistent with the XRD data (FIG. 21C). However, aldehyde treatment has no effect on biomaterial hydration due to low initial mass loss, unless a prior lecithin emulsion is utilized. Lecithin tanned and smoked cellulose biotextiles show an increased initial mass loss of 6.28±1.40%, in comparison to untreated MC. As fabricated MC rapidly decomposes at 340° C. to form a residual mass containing aliphatic compounds. Lecithin, and lecithin tanned and smoked biotextiles show an increased maximum weight loss rate of 58.99±0.55% and 57.77±1.23%, respectively. However, the increased deposition of phosphorus content produces phosphoric acid at high temperatures, facilitating greater foaming char production. The effect of lecithin treatment increased the residual mass to 18.18%, whereas aldehyde treatment reduces char production to 4.61%. Consequently, this suggests that lecithin treatment minimizes the formation of levoglucosan, by lowering the decomposition temperature of cellulose and redirects the path of cellulose decomposition towards greater foaming char formation as an insulating oxygen barrier, resulting in the extreme flame resistance (I. Milosavljevic, V. Oja, E. M. Suuberg, Thermal Effects in Cellulose Pyrolysis: Relationship to Char Formation Processes. Ind. Eng. Chem. Res. 35, 3, 653-662 (1996)).

Flame-retardant chemicals are linked to a myriad of health problems, including autoimmune diseases, learning disabilities, neurological and reproductive problems, birth defects, and cancer (C. M. Butt, J. Congleton, K. Hoffman, M. Fang, H M. Stapleton. Metabolites of Organophosphate Flame Retardants and 2-Ethylhexyl Tetrabromobenzoate in Urine from Paired Mothers and Toddlers. Environ. Sci. Technol. 48, 10432-8 (2014); M. Gascon, M. Fort, D. Martinez, A E. Carsin, J. Forns, J O. Grimalt, L. Santa Marina, N. Lertxundi, J. Sunyer, M. Vrijheid, Polybrominated Diphenyl Ethers (PBDEs) in Breast Milk and Neuropsychological Development in Infants. Environ Health Perspect. 120, 1760-5 (2012)). A study by the Centers for Disease Control and Prevention found that 97% of Americans had flame retardants in their blood, with concentrations higher in children. (Centers for Disease Control and Prevention, “Fourth National Report on Human Exposure to Environmental Chemicals” (2009, cdc.gov/exposurereport/pdf/fourthreport.pdf)). Harnessing microbial biosynthesis to produce a flame retardant, performance biotextile, opens an avenue to mitigate the toxicity of textiles in a range of applications, and to reduce carbon and water footprints throughout a product's circular life cycle.

### Life Cycle Impact Assessment

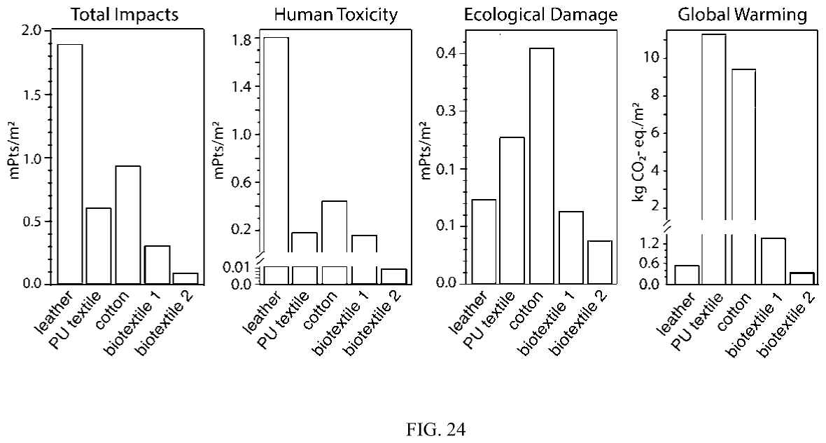

Life Cycle Impact Assessment (LCA) is a quantitative technique to assess the environmental impacts and human health impacts associated with all the stages of a product's life, which is from raw material extraction through materials processing, manufacture, and distribution (M. Z. Hauschild, M. A. J. Huijbregts, “Introducing Life Cycle Impact Assessment” in Life Cycle Impact Assessment. LCA Compendium—The Complete World of Life Cycle Assessment. M. Hauschild, M. Huijbregts, Eds. (Springer, Dordrecht, 2015) pp 1-16. doi.org/10.1007/978-94-017-9744-3_1). FIG. 24 compares the environmental impacts of LT MC biotextiles with the manufacturing impacts of conventional textiles, including chrome tanned leather, synthetic leather (polyurethane (PU)-coated polyester textile), and cotton using a cradle-to-gate. Manufacturing impacts were assessed across impact categories including: ecological damage, comprised of acidification (kg SO₂ eq), ecotoxicity (CTUe), eutrophication (kg N eq), global warming (kg CO₂ eq), and ozone depletion (kg CFC⁻¹¹ eq); human health damage including carcinogenics and non-carcinogenics (both in units of CTUh), respiratory effects (kg PM2.5 eq, fine particulates), and smog ((kg (ground level) O₃ eq), and resource depletion, which refers to extraction of fossil fuels (MJ surplus). For direct comparison, the software converts the impacts to a single score in units of milliPoints (mPts)/m² of textile, utilizing normalization and weighting factors (M. Ryberg, M. D. M. Vieira, M. Zgola, M. J. Bare, R. K. Rosenbaum, Updated US and Canadian Normalization Factors for TRACI 2.1. Clean Techn. Environ. Policy 16, 329-339 (2014)) shown in Table 3, where one point (Pt) represents an average individual's annual share of the total environmental impact in the United States.

LCA results shown in FIG. 24 identify chromium-tanned cow leather as having by far the largest total impact for the textiles considered (1.91 mPts compared to 0.61, 0.94, 0.29 and 0.1 mPts/m² for PU textile, heavyweight cotton, and biotextiles 1 and 2, respectively). Notably, a single impact category, carcinogenics, associated with tanning processes, is responsible for 94.8% of the total impact (1.81 of 1.91 mPts, Table 4). On the other hand, because the Ecoinvent data set available in the software assumes hides are a byproduct of milk and meat production, the inputs do not include the food and land requirements or large climate impacts of the livestock industry (S. J. Kraham, “Environmental Impacts of Industrial Livestock Production” in International Farm Animal, Wildlife and Food Safety Law, G. Steier. K. Patel, Eds. (Springer, Cham. 2017) pp 3-40 doi.org/10.1007/978-3-319-18002-1_1), leather has a minimal carbon footprint (0.59 kg CO₂ eq/m²) compared to synthetic leather and cotton. The global warming impact is greatest for the manufacture of synthetic leather (PU textile, 11.40 kg CO₂ eq/m²), and is the largest impact category (27.5% of the total impacts), followed closely by carcinogens (26.3% of the total impacts). The total impacts for the manufacture of woven, dyed cotton is greater than that of synthetic leather comprised of a PU coating on dyed polyester fabric (0.94 mPts compared to 0.61 mPts) and is about half that of leather (0.94 mPts compared to 1.91 mPts), with the greatest impact categories being carcinogens (40.7%), followed by ecotoxicity (19.5%), and global warming (14.5%).

Biofabrication of MC biotextile 1, which including biosynthesis and processing, has significantly lower total impacts (0.29 mPts) than its conventional textile counterparts (FIG. 24), with an 85% improvement in overall environmental performance improvement compared to leather (Table 4). The largest impact of the biofabrication process is human toxicity (carcinogenics (23.2%) and non-carcinogenics (29.67%), classified as hormonally active agents (HAA)), attributed mainly to refinery processes for sugarcane derived sugar used in the biosynthesis media. To investigate waste-to-resource strategies and minimizing environmental impact, MC biotextiles were also produced by purifying and processing MC obtained as a by-product of commercial kombucha beverage fermentation provided by a local business, OM Champagne Tea (biotextile 2 in FIG. 24). After lecithin treatment, MC biotextiles processed from waste streams (biotextile 2) displayed high tensile strength and flexibility comparable to average values for laboratory biosynthesis (MC biotextile 1, FIG. 26 and Table 1). Elimination of the agricultural impacts of the sugar and tea used as carbon and nitrogen sources in the biosynthesis media, led to a 67% environmental performance improvement for biotextile 2 compared to biotextile 1 (Table 4) and a 97% lower carbon footprint than synthetic leather (0.37 vs 11 kg CO₂ eq./m²). Since fermentation media accounts for ˜30% of the cost of MC production (G. Chen, G. Wu, L. Chen, W. Wang, F F. Hong, L J. Jönsson, Comparison of Productivity and Quality of Bacterial Nanocellulose Synthesized Using Culture Media Based on Seven Sugars from Biomass. Microb. Biotechnol. 12, 677-687 (2019)), and here, the majority of the environmental impact, the flexibility in both choice of carbon and nitrogen sources for the biosynthesis media and the source of MC itself opens exciting opportunities for MC as a platform for sustainable biotextile with both economic and environmental benefit to a circular materials economy.

### Biocoloration and Biodegradation

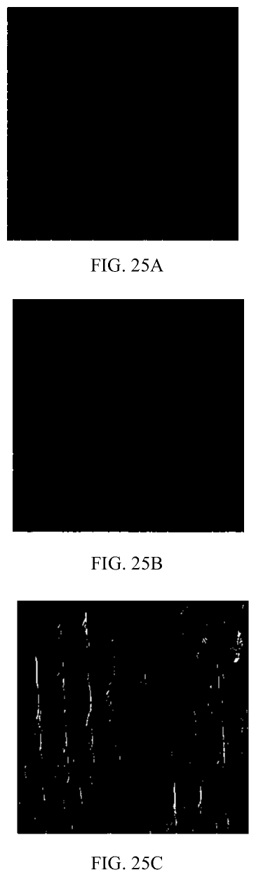

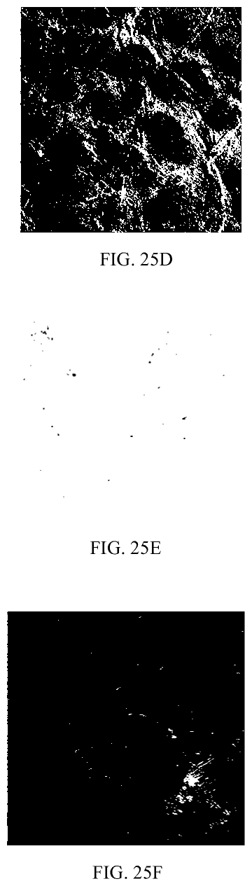

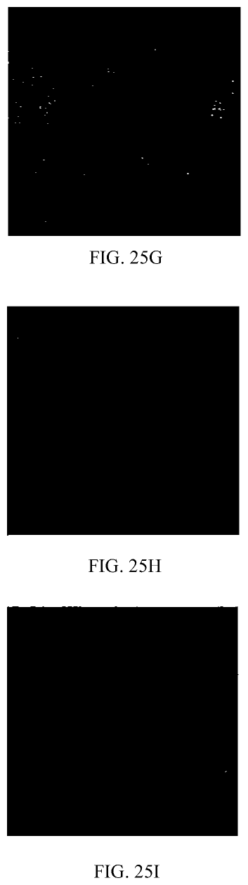

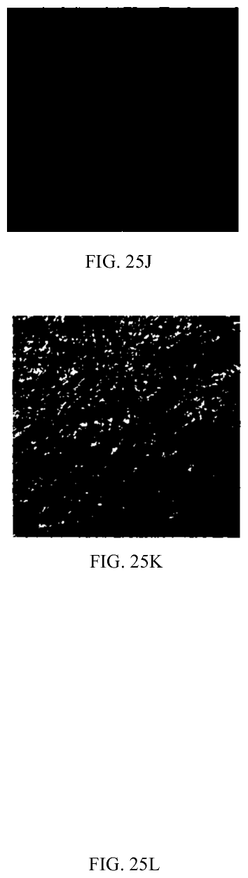

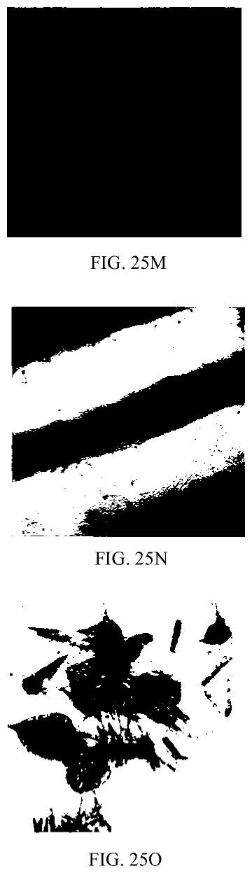

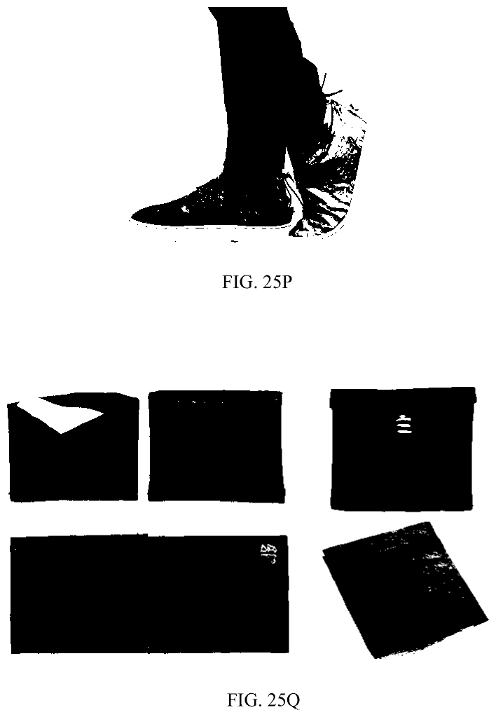

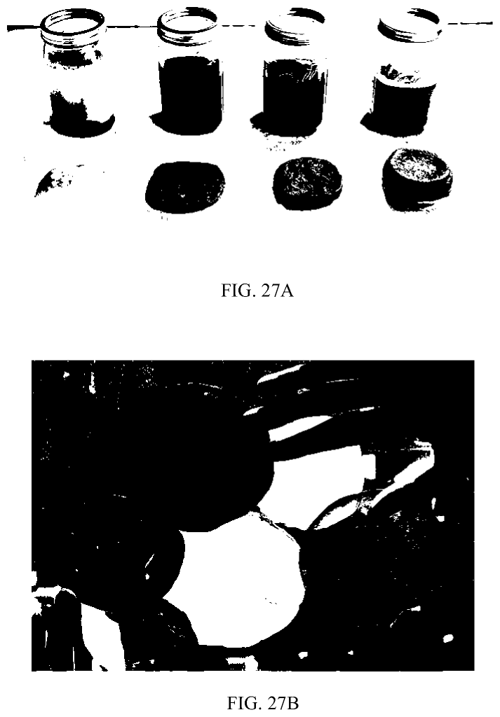

The porosity of MC makes it a good candidate for biocoloration, while the tannic acid provided by the tea used to prepare the media is an excellent natural mordant to bind color (J. E. Song, J. Su, J. Noro, A. Cavaco-Paulo, C. Silva, H R. Kim, Bio-Coloration of Bacterial Cellulose Assisted by Immobilized laccase. AMB Express 8, 19 (2018)). Natural dyes, including important historical dyes such as indigo, madder, and cochineal, were incorporated during biosynthesis (FIGS. 27A-B) or as post-fabrication processing, to create compostable biotextile prototypes with requisite stability and mechanics, in a range of colors and textures shown in FIGS. 25A-O (see Methods for details). Prototypes of footwear and accessories, including “bioleather” sneakers and wallets, constructed using an industrial sewing machine, are also shown. Using data from Murugesh et al., (K. B. Murugesh, Life Cycle Assessment for the Dyeing and Finishing Process of Organic Cotton Knitted Fabrics, JTATM, 8, 2 (2003)) the inventors calculate that biocoloration of 1 m² of cellulose-based textile (400 g) with yellow onion skins, obtained as food by-product (FIG. 25H), with color extracted as part of media preparation, would eliminate 348.13 kg TEG ecotoxicity, 0.18 kg C₂H₃Cl eq. human health, and 5.4 kg CO₂ eq. of climate impacts from synthetic dyeing and finishing.

To investigate the compostability of the MC biotextiles, LT MC samples (n=5) were weighed and buried at least 2.5 cm deep in vessels of nutrient rich soil free (pH=6.9, Nitrate, K and P levels of X, Y, Z). After 60 days outdoors, with average high and low temperatures of 14.2 C and 3.2 C respectively, samples were retrieved and weighed. LT MC samples showed significant visible deterioration, were smaller in size, and crumbled easily, and had lost 74.45+/−2.94% of initial mass.

### Conclusions

Once treated, the BC biotextile is water resistant and durable. The treated MC is compostable but the treatment enhances the durability and resistance to chemical degradation and decomposition, outside of a microbial rich environment such as a compost pile or bin at the end its useful product life. Note that this cradle-to-gate LCA does not include end of life impacts, which is of particular concern for plastic-based textiles, such as synthetic leather.

### Methods for Experimental Set 2

### Preparation of Microbial Cellulose Pellicles

Microbial cellulose pellicles were prepared in culture media containing 5.8 w/v % sucrose, 2% w/v green tea as a nitrogen source, and Symbiotic Colony of Bacteria and Yeast (SCOBY) obtained from Fermentaholics© or provided by OM Champagne Tea, a commercial kombucha beverage facility. Culture media was inoculated with 10 w/v % SCOBY starter culture consisting of a combination of bacteria and yeast. Static fermentation at room temperature was maintained until a pellicle at least 2 cm thick was formed at the air-liquid media interface. Biofabrication proceeds as 2-D layer by layer production of a cellulose biofilm which takes the shape of the fermentation vessel. Formed pellicles were washed three times with deionized water to remove residual sugars and air dried for 72 hours at room temperature.

As a post-production treatment, hydrated pellicles were immersed in a lecithin emulsion for 24 hour (LT MC). The lecithin emulsion consisted of 5% w/v lecithin powder (soy or sunflower seed) created as a byproduct of the edible oil industry, and a 20% v/v sunflower seed oil in water and blended at high speed for 60 seconds. An emulsified solution of phospholipids and enzymes, derived from mammalian tissue or sunflower seed oil.

Dehydrated MC samples treated with and without lecithin tanning, were then exposed to an additional smoke tanning aldehyde treatment, in which biofilms were placed into a hydrocarbon-rich environment for 1 hour, using a Smoke Hollow 26142E 26-Inch Electric Smoker with Adjustable Temperature Control and Kingsford smoking wood chips, with the temperature between 175 and 220 F. Prolonged exposure of the MC biotextiles to hydrocarbon-rich smoke seals in the lecithin and oils.

### Characterization

Microbial cellulose morphology was assessed by using a scanning electron microscope (Zeiss Sigma VP, Oberkochen, Germany; 3 kV; n=5). Briefly, hydrated microbial cellulose samples were initially placed in a −20° C. freezer for 24 hours and lyophilized in a freeze dryer system (Labconco FreeZone, Kansas City, Mo., USA) for 24 hours at −84° C. and 2.0×10⁻² mbar. Prior to imaging, samples were sputter coated (Cressington 108, Watford, UK) with 30 nm of gold. The fiber diameter of each sample was measured by analyzing randomly selected fiber segments in SEM images using NIH ImageJ software (Bethesda, Md., USA; n=100), to calculate an average fiber diameter. Biotextile hydrophilicity was measured via water contact angle (WCA) measurements. WCA is the angle formed tangential to the water droplet at the air-liquid-solid interface. A WCA <90 degrees indicates a hydrophilic surface; a WCA greater than 90 degree indicates a hydrophobic surface. A dropper containing distilled water was secured 1 cm above the sample surface and the contact angle of the water droplet on the surface was measured. Mechanical properties of microbial cellulose biotextiles were assessed by securing samples with custom clamps and mounting in a uniaxial tensile testing machine (Instron, Model 1321, Norwood, Mass., USA), equipped with a 25 kN load cell. Biotextiles were maintained to have a gauge length of 2 inches and were tested to failure. Microbial cellulose elastic modulus, toughness, and ultimate tensile strength were determined from the stress-strain curve.

### X-Ray Diffraction

A 5-10 mm punch pressed into each of the various treated samples to create a series of small disks. The disks were placed on a standard glass slide. Diffractograms were collected using a Panalytical XPert3 Powder diffractometer with a 3 kW generator, fully ceramic Cu Long fine focus (LFF), X-ray tube, vertical goniometer (theta-theta) and a PIXcel 1d detector. The copper long fine focus lens produces a k alpha of 1.5406 angstroms. Measurements were collected while scanning from 5 to 100 degrees with the following parameters: Fixed divergence slit: ½ degree, step size: 0.026 deg/step, scanning rate: 10.7 deg/min. No background correction was performed.

The lattice spacing (d-spacing) was calculated using Bragg's equation:

λ=2dₕₖₗ*sin θ

where dₕₖₗ is the lattice spacing of the crystallographic planes, θ is the corresponding Bragg angle, and λ is the X-ray wavelength (0.154 nm).

The Segal Crystallinity Index (CI) was calculated:

$${C}_{I} = \frac{{I}_{t} - {I}_{a}}{{I}_{t}}$$

as used by Nam et al. (S. Nam, A. French, B. Condon, M. Concha, Segal crystallinity index revisited by the simulation of X-ray diffraction patterns of cotton cellulose I and cellulose II. Carbohydr. Polym. 135, 1-9 (2016)). The total intensity Iₜ was taken for the (002) peak at 22.7° and the amorphous intensity was taken from the amorphous region represented at 18.3° which is the local minima between the (002) and (101) peaks.

### Thermal Analysis

A 45° Flame Test (ASTM D1230-94), which is a widely-accepted method for determining apparel textiles flammability, was used to measure: 1) Ease of textile ignition, and the 2) Duration of flame spreading. Biotextiles (1″×18″ in FIGS. 23A-D) were mounted and held in a custom apparatus at an 45° angle. A standardized flame was applied to one end of the biotextile at a 45° angle using 14.1 oz. Map-Pro Cylinder by Bemzomatic, which uses liquid propane as a fuel source and has a flame temperature in air of 3,730° F./2054.4° C.

Charred biotextile surfaces were observed with SEM. Thermal properties and char formation of dried, pre-weighed microbial cellulose discs (diameter: 4.5 mm) were determined by a thermogravimetric analyzer (TGA 550, TA Instruments; New Castle, Del.). TGA analysis was performed at a heating rate of 10° C./min over the temperature range of 25° C. to 700° C. under flowing nitrogen (40 mL/min), in which final weights indicated the formation of char residue.

Chemical and elemental analysis via FTIR, XPS, and EDAX were performed to support microbial cellulose mechanical, hydrophilic, and thermal characteristics.

FTIR (LUMOS II, Bruker, Billerica, Mass., USA) spectra were recorded under attenuated total reflectance (ATR) mode in a spectral range of 4000-600 cm⁻¹. Each spectrum was collected using a total of 200 scans and a resolution of 4 cm⁻¹. Crystallinity analysis of the biotextiles by FTIR spectrums was performed, according to empirical formulas proposed by Nelson and O'Connor. While the ratio between absorbances at 1429 cm⁻¹ to 897 cm⁻¹ is defined as the lateral order index (LOI) for assessing cellulose's overall degree of order, the ratio between 1372 cm⁻¹ to 2900 cm⁻¹ is defined as total crystallinity index (TCI), which is related to the degree of crystallinity in cellulose. The formulas used for these calculations are as follows:

Chemical bonding states of microbial cellulose biotextiles were determined by XPS (PHI 5500, Chanhassen, Minn., USA). The spectra were recorded using a monochromatic Mg-Ka radiation X-ray source (1253.6 eV) and the analyzer pass energy was set to 25 eV. The sample chamber was set to 50 W operating at 15 kV voltage and a base pressure of 2×10⁸ torr. The XPS spectra were collected in the range from 0 to 1200 eV, with a resolution of 0.1-1.0 eV. The inelastic background of the C1s, O 1s, N1s, and P 2p electron core spectra was subtracted using Shirley's method and data was analyzed using commercial curve fitting software Igor64 (WaveMetrics, Portland, Oreg., USA). The binding energy scale was calibrated using the Au 4f 4/7 line of 10 nm thick gold, electron beam evaporated into 5 mm wide strips onto two parallel edges of the LT and MC samples, and confirmed against the C 1s binding energy for adventitious carbon on Au (285 eV) as well as published XPS data for microbial and plant cellulose. Based on these values, MC and LT XPS data were shifted −1.98 and −2.88 eV to higher binding energy, respectively. A smoothing factor of 10 was applied to the P 2p XPS data for ease of comparison (no smoothing was applied to C 1s or O 1s data).

Further surface elemental characterization was assessed by an EDS system (Bruker XFlash 6, Billerica, Mass., USA; 10 kV; 5 minutes; n=3) connected to a scanning electron microscope (Zeiss Sigma VP, Oberkochen, Germany). Biotextiles were sputter coated (Cressington 108, Watford, UK) with copper, and assessed for the presence of phosphorus.

### Life Cycle Impact Assessment (LCA)

LCA analysis is a quantitative technique to assess environmental impacts associated with all the stages of a product's life, which is from raw material extraction through materials processing, manufacture, distribution, and use as specified in ISO 14040 standards (International Organization for Standardization (ISO), Environmental Management—Life-Cycle Assessment-Principles and Framework, International Standard 14040; ISO: Geneva, Switzerland, 2006; International Organization for Standardization (ISO), Environmental Management—Life Cycle Assessment—Requirements and Guidelines, International Standard 14044; ISO: Geneva, Switzerland, 2006; Muralikrishna, I. V., & Manickam, V. (2017). Life Cycle Assessment. Environmental Management, 57-75, doi.org/10.1016/b978-0-12-811989-1.00005-1). Manufacturing impacts including ecological damage, human health damage, climate impacts and resource depletion were determined using Sustainable Minds® Life Cycle Assessment software (Cambridge, Mass., USA) and the EcoInvent database (Zurich, Switzerland). The impact categories are based on the U.S. EPA's Tool for the Reduction and Assessment of Chemical and other Environmental Impacts (TRACI) life cycle impact assessment (LCIA) methodology. As defined by the software, ecological damage is comprised of acidification (kg SO₂ eq), ecotoxicity (CTUe), eutrophication (kg N eq), global warming (kg CO₂ eq), and ozone depletion (kg CFC⁻¹¹ eq). Human health damage includes carcinogenics and non-carcinogenics (both in units of CTUh), respiratory effects (kg PM2.5 eq, fine particulates), and smog ((kg (ground level) 03 eq), while resource depletion exclusively refers to extraction of fossil fuels (MJ surplus).

Impacts were calculated in a cradle-to-gate LCA comparing the impacts of biofabrication microbial biotextiles using the methods described above with the manufacturing impacts of conventional textiles, including chrome tanned leather, synthetic leather (polyurethane (PU)-coated textile), and cotton canvas. For direct comparison, impacts were calculated from weighted impact categories, in mPts/m² of textile, utilizing normalization and weighting factors shown in Table 5. A point (1000 mPts) represents the average person's annual environmental load (i.e., entire production/consumption activities in the economy) in the United States {http://www.sustainableminds.com/showroom/shared/learn-single-score.html}. Normalization factors were calculated using characterization factors from the TRACI 2.1 LCIA model, and toxicity-based categories use characterization factors calculated with USEtox. Weighting factors represent degree of immediate concern or degree to which remedial actions associated with the impact are underway, and provide a practical method to link quantitative results of LCA with environmental performance of competing products and assist environmentally preferable design, manufacture, and purchasing (Ry berg, M., Vieira, M. D. M., Zgola, M. et al. Updated US and Canadian normalization factors for TRACI 2.1, Clean Techn Environ Policy 16, 329-339 (2014); Weighting: Gloria, T. P.; Lippiatt, B. C.; Cooper, J. Environ. Sci. Technol. 2007, 41, 21, 7551-7557). A cradle-to-gate partial product life cycle was performed from resource extraction (cradle) to the factory gate which includes raw material and manufacturing impacts, with distribution, consumer use and disposal omitted; such assessments are often used as the basis for environmental product declarations (EPD).

### LCA: Synthetic Leather

Synthetic leather (polymer coated textile) can be produced with a variety of synthetic polymers, including polyvinyl chloride (PVC), polyvinylidene chloride (PVDC), polyurethane (PU), and different base fibers (cotton, polyester or cotton/synthetic blends), and coating techniques. Each one of these variables affects the ecological impact of synthetic leather. The variation chosen for this LCA is PU-coated polyester made by the company Kuraray, specifically their product called Clarino Crust. According to market research, this enterprise is one of the main manufacturers of synthetic leather, and the material data sets in the Ecolnvent database are, in fact, based on the specific manufacturing processes used by Kuraray (Synthetic Leather (Artificial Leather) Market by Type (Polyurethane, Polyvinyl Chloride, Bio-based), End-Use Industry (Footwear, Furnishing, Automotive, Clothing, Bags, Purses & Wallets, Sports, Electronics)—Global Forecast to 2021. marketsandmarkets.com/Market-Reports/synthetic-leather-market-6616309.html). According to Kuraray's Clarino Crust technical sheet, the weight of the textile is 0.66±0.02 kg/m² and contains 90% of polyester (PET)—0.594 kg—and 10% polyurethane (PU) −0.066 kg—It is important to note that the only information taken from the Clarino Crust technical sheet (Status September 2018. clarino.eu/fileadmin/user_upload/CLARINO/technical_data_sheets/clarino_crust/Clarino_Cr ust.pdf) is the weight and the percentage—of PU and PET-utilized. Subsequently, the material inputs for PU flexible foam included in the Ecolnvent database within Sustainable Minds, CAS number: 009009-54-5, was provided by Kuraray and contains the transport of the monomers, the production (energy, air emissions) of the PU foam, and using average values of transport and infrastructure, for the typical composition for European conditions and present technology used in Europe.

The polyester inputs were based on average data for dyed, manufactured polyester, and not provided by a particular brand, and the processing for PU foams chosen was foaming as it is to our knowledge the best option available. The process of foaming by expanding plastic involves converting the plastic foam into an emulsion and then coating the base fabric (A. K. Sen, Coated Textiles—Principles and Applications, 2nd Edition, CRC Press, Boca Raton, Fla., USA, 2008. (P. 64; 147)). The process data set included the auxiliaries and energy demand for the conversion process of plastics and the converted amount of plastics was not included into the dataset. The complete system of materials used to model the manufacturing of PU-coated polyester was obtained from the Ecoinvent database.

### Cow Leather

Material inputs for the manufacture of cow leather tanned with a chromium process from the Ecoinvent database. According to the database, the inventory assumes the hides are a byproduct of milk and meat production without significant value, and therefore neglects the inputs of 989 kg of food and 0.56 ha of grassland per cow, based on average data from the Netherlands. The functional unit is 1 m², corresponding to a measured mass of 0.4 kg.

### Cotton

The Ecoinvent data set used for assessing the manufacturing impacts of cotton is based on average data for a dyed, manufactured woven cotton, and not provided by a particular brand. Mass measurement 1 m² of cotton (0.337 kg) was used in the comparison. Data set inventory details are as follows: US-ecoinvent 2.2 Name: 1 lb Sodium sulfate, anhydrite {GLO}| textile production, knit cotton, yarn dyed|Alloc Def, U (of project Ecoinvent 3—allocation at point of substitution—unit) Geography: China, Eastern Europe based on largest manufacturing regions. Included processes: This process links the processes yarn production and weaving.

### Microbial Cellulose Biotextiles

Biotextile 1: Laboratory scale biosynthesis and processing (lecithin tanning (LT) of 1 m² microbial biotextile. The system bill of materials was created from the Ecoinvent data sets. The biosynthesis cultivation media to biofabricate 1 m² of microbial cellulose included 1.74 kg of sugar, 120 grams of green tea, 30 liters of water and 3864.68 Btu of heat energy from a natural gas boiler needed to raise the temperature of 15 liters of water from 25 C to 90 C (Q==mcΔT) to steep the tea and dissolve the sugar used in the cultivation media. Half the total water use of the liquid media (15 liters) is heated, the other half is added at room temperature to cool the media to a sufficiently low temperature (38 C or less) before adding 10% v/v starter culture to the media. The Ecoinvent data set for sugar included production of sugar from sugarcane and manufacturing at the sugar refinery. Impacts for production of 120 grams of tea used to produce 1 m² microbial cellulose were calculated based on APOS, U (of project Ecoinvent 3 {LK}) data for 1 kg tea, which includes farm cultivation, including irrigation, land use, field spraying insecticides and fertilizers (ammonium nitrate, ammonium sulfate, glyphosate, diammonium phosphate production, drying and processing. Material inputs for the lecithin tanning emulsion (LT) included 0.28 kg of sunflower oil and 2 liters of distilled water to prepare the emulsion, and another liter of water to rinse after tanning. The sunflower seed oil data set included sunflower seed production and oil mill process. Phosphatidylcholine (lecithin powder) is produced as a byproduct from the edible oil industry, and as such, impacts are considered accounted for in the data set for sunflower seed oil, and is not available as a separate material input.

Biotextile 2: Purification and Processing (LT) of commercial kombucha fermentation by-product (symbiotic colony of bacteria and yeast (SCOBY)). The system bill of materials includes purification and processing (LT described above) of commercial kombucha fermentation by-product (SCOBY) to produce 1 m² of microbial cellulose. The system of materials was based on the Ecoinvent data set and shown in Table 3. In the purification process of the SCOBY provided by OM Champagne Tea, 8 liters of water and the heat energy to rise to 85 C were utilized. The material inputs for the lecithin tanning process included 200 ml sunflower seed oil and 2 liters of water to tan 1 m² of bioleather.

### Rationale for LCA Comparison: Market Data of the Leather and Synthetic Leather Goods Industry.

Both synthetic and animal leather are frequently utilized within the apparel, automotive, and furnishing industries. According to Grand View Research (Synthetic Leather Market Size, Share & Trends Analysis Report By Product (Bio-based, PVC, PU), By Application (Clothing, Furnishing, Automotive, Bags & Wallets, Footwear), By Region, And Segment Forecasts, 2020-2027 grandviewresearch.com/industry-analysis/synthetic-leather-market). The global synthetic leather market size was valued at USD 29.3 billion in 2019 and is expected to increase to USD 30.3 billion for 2020. It is also estimated that the market size volume in 2020 is going to account for 15,585.6 Million Meters. The market size value of animal leather is estimated to have been over USD 80 billion in 2019 (International Trade Centre, intracen.org/itesectors/leather). As reported by Grand View Research, the textile market size value in 2020 is USD 1,000.30 billion. Subsequently, cotton was anticipated to be the largest raw material segment, accounting for a market share of 39.5% in 2019 (Market Size, Share & Trends Analysis Report By Raw Material (Wool, Chemical, Silk, Cotton), By Product (Natural Fibers, Polyester, Nylon), By Application, By Region, And Segment Forecasts, 2020-2027—.grandviewresearch.com/industry-analysis/textile-market).

### Biocoloration of Microbial Cellulose Biotextiles—Natural (Plant and Mineral) Dyeing

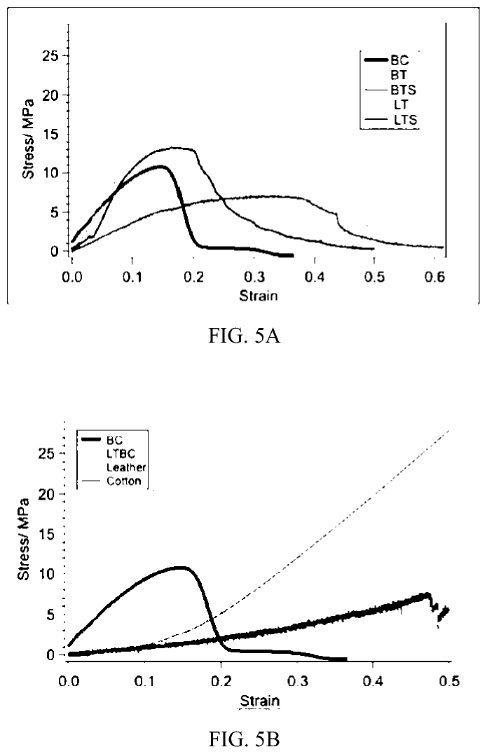

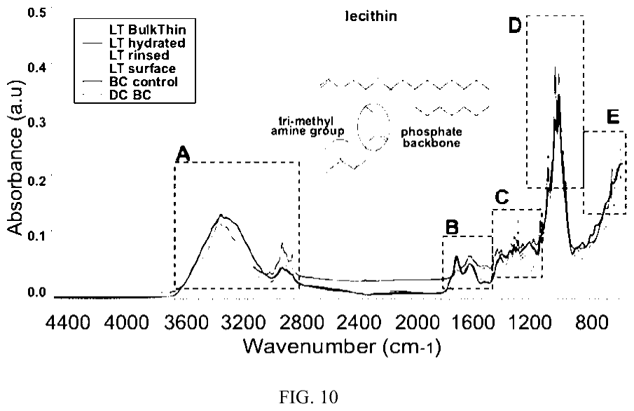

To reduce the water and energy demands of traditional dip dye methods, natural dye matter was added with tea and sugar for coincident extraction of color and preparation of the cultivation media and strained before adding bacteria inoculum to the media—so that color could be directly incorporated into the nanofiber mesh during biosynthesis. The tannins in the tea serve to mordant the cellulose fibers, to improve color retention, saturation and colorfastness. Cultivation vessels were prepared with 5.3% w/v sugar, 4% w/v tea, 10% v/v starter culture and natural dye matter in 7.5% w/v cochineal (FIG. 25A); 20% w/v (FIG. 25B) and 10% w/v (FIG. 25C) madder root 9.75% w/v marigold flowers (FIG. 25D) and 5.5% w/v marigold extract (FIG. 25E); yellow onion skins 3% w/v (FIG. 25F); 7.5% v/v liquid chlorophyll (FIG. 25G). Onion skins were obtained as food by-product and chlorophyll was obtained from Horb\dot{a}ach, Melville, N.Y. All other dye stuff was obtained from Botanical Colors, Seattle, Wash.

For red shown in FIG. 25A and FIG. 20, 10 grams of cochineal were ground to a fine powder, placed in a cotton muslin pouch and tied with cotton string, and immersed in 250 ml distilled water and brought to 100 C for 15 minutes. The heat was turned off, allowed to sit for 15 minutes, after which the process of adding water and bringing to a boil was repeated 3 times. On the last step the tea and sugar for the culture media were added, after which the cochineal pouch was removed, the additional water for the culture media was added, cooling the media to a temperature suitable to add the inoculum (10% v/v). Other colors were extracted in one liter of water held at 85 C to 45-1 hour, except for chlorophyll, which was added with the inoculum to the media.

Post synthesis dip dyeing in room temperature organic indigo vats, produced from combining 5 g organic indigo powder, 10 g calcium hydroxide and 15 g fructose in 15 liters of water were used to create greens (FIG. 25L) and blues (FIG. 25M). White biotextiles were achieved by immersion of hydrated MC in 0.5 M NaOH at room temperature for 12 hours, followed by rinsing in distilled water until a neutral pH was reached (FIG. 25I). Black biotextiles were achieved by chemical reaction of tannic acid and FeSO₄. Samples were immersed in a 3% w/v tannic acid solution, obtained from Botanical Colors, followed by a dip in aqueous FeSO₄ mordant, prepared by soaking 100 grams of nails in 2 liters of 2.5% v/v acetic acid for two weeks.

### Biodegradation Studies

Dry 20 mm×20 mm samples (n=5) of as grown and lecithin tanned microbial cellulose were weighed and buried in pots containing soil, pH 6.9, with Nitrate, P, and K levels of X, Y, and Z, respectively, as measured with a Rapid Soil Analysis test (model number). After 60 days outdoors; with average high and low temperatures for the 60 day period were 14.2 and 3.2 C, respectively, samples were removed and weighed.

### Statistical Analysis

All quantitative values are reported as means±standard deviation, with n equal to the number of replicates per group. Two-way analysis of variance (ANOVA) and the Tukey-Kramer post-hoc test was used for all pairwise comparisons, and significance was attained at p<0.05. Statistical analyses were performed with JMP-IN (4.0.4, SAS Institute, Inc., Cary, N.C., USA).

### Tables

TABLE 1
Mechanical properties (Young’s Modulus, Toughness and Max Strength), crystallinity index, fiber
diameter, and water contact angle of microbial cellulose as fabricated (MC) and after various
treatments (LT, LTS, S) corresponding to data shown in FIG. 20, compared to cotton and leather.

| | Young's | | Max | | Fiber | | Contact |
|---|---|---|---|---|---|---|---|
| | Modulus | Toughness | Strength | Crystallinity | Diameter | | Angle (°; |
| Sample | (MPa; n = 4) | (MPa; n = 4) | (MPa; n = 4) | Index (CI) | (nm; n = 100) | | n = 5) |
| MC1 | 210.91 ± 58.61 | 2.48 ± 0.61 | 17.41 ± 4.64 | 60.93 | 71.00 ± 19.00 | | — |
| MC2 | 58.30 ± 35.71 | 2.44 ± 1.44 | 11.96 ± 5.76 | | | | — |
| LT | 196.44 ± 65.42 | 7.15 ± 2.26 | 27.91 ± 1.75 | 74.43 | 69.35 ± 16.79 | | — |
| LTS | 89.88 ± 45.20 | 2.23 ± 0.31 | 11.10 ± 1.47 | 45.74 | 76.54 ± 16.17 | | — |
| S1 | 38.89 ± 19.11* | 0.14 ± 0.05* | 3.35 ± 1.47* | 39.86 | 56.58 ± 14.61 | | — |
| S2 | 23.99 ± 3.34* | 1.51 ± 0.98* | 5.52 ± 2.75* | | | | — |
| cotton | 19.66 | 2.60 | 8.01 | — | 12.70 ± 3.11 | μm | — |
| leather | 75.88 | 5.35 | 28.42 | | 20-200 | μm* | — |

*Xinxin Li, Ya-nan Wang, Jing Li and Bi Shi, JALCA, VOL. 111, 2016, 230-237.
\hat{ } indicates significance between groups (α = 0.05, n = 5).

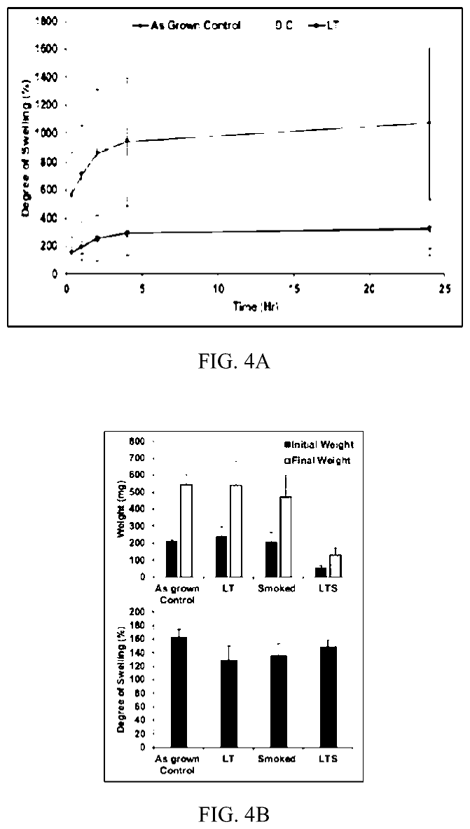

TABLE 2
Thermal decomposition regions of TGA data (FIG. 4D) for as fabricated
(MC) and processed (S, LT, LTS) microbial cellulose.

| | | | | Residual Mass |
|---|---|---|---|---|
| | Mass Loss (%) | | | at 680° C. (%) |
| Sample | Regions | | | |
| (n = 5) | I | II | III | IV |
| MC | 5.38 ± 0.87 | 16.96 ± 2.37 | 49.51 ± 1.21 | 8.99 |
| LT | 8.50 ± 0.25 | 29.55 ± 0.25 | 58.99 ± 0.55 | 18.18 |
| S | 4.15 ± 0.91 | 19.88 ± 3.76 | 50.42 ± 1.49 | 4.61 |
| LTS | 6.28 ± 1.40 | 28.60 ± 2.58 | 57.77 ± 1.23 | 15.45 |

TABLE 3
Lateral order index obtained from FTIR data showing
increase in crystallinity with lecithin tanning
(LT) compared to as-fabricated MC.

| Lateral Order Index | MC | LT |
|---|---|---|
| LOI (A₁₄₂₉/A₈₉₇) | 0.84848485 | 0.91044776 |

TABLE 4
Mechanical properties obtained from tensile testing data shown in FIG. 26 for microbial cellulose
obtained as kombucha brewery by-product, after purification and lecithin tanning (n = 5).

| | Thickness | Width | Area | Young's | Max Stress | Yield Strength | Resilience | Toughness |
|---|---|---|---|---|---|---|---|---|
| Sample | (mm) | (mm) | (mm\hat{ }2) | Modulus (MPa) | (MPa) | (MPa) | (MPa) | (MPa) |
| 1 | 1.55 | 12.70 | 19.69 | 213.71 | 17.13 | 12.21 | 0.37 | 3.75 |
| 2 | 1.38 | 12.70 | 17.53 | 341.44 | 30.21 | 20.59 | 0.67 | 7.69 |
| 3 | 1.60 | 12.70 | 20.32 | 172.59 | 15.17 | 12.34 | 0.47 | 4.95 |
| 4 | 1.52 | 12.70 | 19.30 | 271.54 | 22.95 | 15.50 | 0.49 | 7.44 |
| 5 | 1.19 | 12.70 | 15.11 | 298.06 | 28.90 | 19.44 | 0.70 | 6.39 |
| Avg | 1.45 | 12.70 | 18.39 | 259.47 | 22.87 | 16.02 | 0.54 | 6.04 |
| STD | 0.17 | 0.00 | 2.1 | 67.08 | 6.76 | 3.90 | 0.14 | 1.67 |

TABLE 5
LCA normalization and weighting factors used to convert
specific impacts to an impact factor expressed in mPts per
functional unit (here 1 m² of textile).

| | Normal- | | Weighting |
|---|---|---|---|
| Impact category | ization | Unit | (%) |
| Ozone depletion | 1.61E−01 | kg CFC⁻¹¹ eq/year/capita | 2.40E+00 |
| Global warming | 2.42E+04 | kg CO₂ eq/year/capita | 3.49E+01 |
| Smog | 1.39E+03 | kg O₃ eq/year/capita | 4.80E+00 |
| Acidification | 9.09E+01 | kg SO₂ eq/year/capita | 3.60E+00 |
| Eutrophication | 2.16E+01 | kg N eq/year/capita | 7.20E+00 |
| Carcinogenics | 5.07E−05 | CTUh/year/capita | 9.60E+00 |
| Non-carcinogenics | 1.05E−03 | CTUh/year/capita | 6.00E+00 |
| Respiratory effects | 2.43E+01 | kg PM2.5 eq/year/capita | 1.08E+01 |
| Ecotoxicity | 1.10E+04 | CTUe/year/capita | 8.40E+00 |
| Fossil fuel depletion | 1.73E+04 | MJ surplus/year/capita | 1.21E+01 |

TABLE 6
Cradle to Gate LCA - Manufacturing Impacts for 1 m² of textile. Data values
contained in this spreadsheet are based on calculations from within the
Sustainable Minds Software using the Ecoinvent data sets described.

| Textile: | Leather | PU textile | Cotton | Biotextile 1 | Biotextile 2 |
|---|---|---|---|---|---|
| Mass of 1 m² (kg) | 0.4 | 0.6 | 0.34 | N/A | N/A |
| Material | Cow leather | Dyed polyester | Dyed, | Water (40 l) | Water (10 l) |
| Inventory | (chromium | (0.594 kg) | Woven | Green tea (120 g) | Sunflower |
| | process) | PU flexible foam | Cotton | Sunflower | Seed Oil (10 oz) |
| | | (0.066 kg) | | Seed Oil (296 ml) | Natural Gas |
| | | | | and Sugar (1740 g) | Heat |
| | | | | Natural gas heat | |
| Additional | N/A | Foam expansion | N/A | N/A | N/A |
| Processes | | plastics | | | |
| Impact category | | | | | |
| Total Impacts (mpts) | 1.91 | 0.61 | 0.942 | 0.295 | 9.63E−02 |
| Acidification | 1.0E−03 | 3.2E−02 | 3.4E−02 | 2.74E−03 | 5.75E−04 |
| Ecotoxicity | 7.3E−02 | 3.0E−02 | 0.183 | 4.88E−02 | 2.05E−02 |
| Eutrophication | 2.0E−04 | 2.8E−02 | 6.0E−02 | 5.55E−02 | 5.06E−02 |
| Global warming | 8.0E−03 | 0.165 | 0.136 | 1.3E−02 | 5.29E−03 |
| Ozone depletion | 9.69 | 1.0E−04 | 3.4E−05 | 1.75E−05 | 8.28E−06 |
| Fossil fuel depletion | 6.9E−03 | 0.118 | 2.4E−02 | 9.42E−03 | 4.09E−03 |
| Carcinogenics | 1.81 | 0.16 | 0.38 | 6.85E−02 | 7.07E−03 |
| Non-carcinogenics | 1.4E−03 | 2.2E−02 | 6.3E−02 | 8.75E−02 | 7.00E−03 |
| Respiratory effects | 7.0E−04 | 1.0E−02 | 3.5E−02 | 6.67E−03 | 1.00E−03 |
| Smog | 9.0E−04 | 4.4E−02 | 2.3E−02 | 2.80E−03 | 8.06E−05 |
| CO₂ eq. (kg) | 0.58 | 11.40 | 9.46 | 0.89 | 0.37 |

TABLE 7
Impacts for 1 m² of polyurethane (PU)-coated polyester
textile (synthetic leather) by input.

| Input | Polyester (PET) | PU flexible foam | Foaming |
|---|---|---|---|
| Total impacts | 0.58 | 2.83E−02 | 2.54E−03 |
| Acidification | 3.08E−02 | 5.56E−04 | 1.49E−04 |
| Ecotoxicity | 2.86E−02 | 1.73E−03 | 1.55E−04 |
| Eutrophication | 2.60E−02 | 1.56E−03 | 2.61E−05 |
| Global warming | 0.159 | 4.64E−03 | 8.47E−04 |
| Ozone depletion | 1.27E−04 | 2.64E−07 | 9.44E−07 |
| Fossil fuel depletion | 0.113 | 4.85E−03 | 3.98E−04 |
| Carcinogenics | 0.145 | 1.27E−02 | 5.45E−04 |
| Non-carcinogenics | 2.10E−02 | 8.54E−04 | 1.76E−04 |
| Respiratory effects | 8.41E−03 | 9.37E−04 | 1.08E−04 |
| Smog | 4.30E−02 | 5.64E−04 | 1.38E−04 |
| CO₂ eq. (kgs) | 11.0 | 0.32 | 5.88E−02 |

TABLE 8
Cradle to Gate LCA -Biotextile 1- Impacts for biosynthesis
and lecithin tanning of 1 m² of LT MC textile by input.

| | | Sunflower | Green | Natural | Tap |
|---|---|---|---|---|---|
| Input | Sugar | Seed Oil | Tea | gas | Water |
| Impacts (mpts) | 1.74 kg | 296 ml | 120 g | 3864.7 Btu | 401 |
| Total Impacts | 0.192 | 1.92E−02 | 1.24E−04 | 1.04E−02 | 1.34E−03 |
| Acidification | 2.08E−03 | 5.17E−04 | 1.42E−06 | 1.04E−04 | 4.32E−05 |
| Ecotoxicity | 2.82E−02 | 2.05E−02 | 5.50E−05 | 4.99E−05 | 6.85E−05 |
| Eutrophication | 4.85E−03 | 5.06E−02 | 1.15E−05 | 3.4E−05 | 8.51E−06 |
| Global warming | 5.08E−03 | 3.31E−03 | 2.95E−06 | 4.23E−03 | 2.51E−04 |
| Ozone depletion | 5.12E−6 | 4.92E−6 | 2.43E−09 | 7.38E−06 | 1.29E−07 |
| Fossil fuel depletion | 2.65E−03 | 1.91E−03 | 1.40E−06 | 4.79E−03 | 7.28E−05 |
| Carcinogenics | 6.04E−02 | 6.49E−03 | 1.95E−05 | 8.77E−04 | 7.17E−04 |
| Non carcinogenics | 8.04E−02 | 6.93E−03 | 3.08E−05 | 1.01E−04 | 1.13E−04 |
| Respiratory effects | 5.61E−03 | 9.64E−04 | 1.21E−06 | 6.07E−5 | 3.65E−05 |
| Smog | 2.61E−03 | 1.11E−06 | 5.55E−07 | 1.59E−04 | 3.10E−05 |
| CO₂ eq. (kg) | 0.353 | 0.229 | 2.05E−02 | 0.294 | 1.74E−02 |

TABLE 9
Cradle to Gate LCA -Biotextile 2 - Impacts
for 1 m² of biotextile by input.

| | Tap | Natural gas (boiler | Sunflower |
|---|---|---|---|
| Input | water | modulating >100 kW) | Seed Oil |
| Total Impacts | 3.35E−04 | 4.71E−03 | 9.12E−02 |
| Acidification | 1.08E−05 | 4.67E−05 | 5.17E−04 |
| Ecotoxicity | 1.71E−05 | 2.25E−05 | 2.05E−02 |
| Eutrophication | 2.13E−06 | 1.54E−05 | 5.06E−02 |
| Global warming | 6.27E−05 | 0.00192 | 3.31E−03 |
| Ozone depletion | 3.22E−08 | 3.33E−06 | 4.92E−6 |
| Fossil fuel depletion | 1.82E−05 | 2.16E−03 | 1.91E−03 |
| Carcinogenics | 1.79E−04 | 3.96E−04 | 6.49E−03 |
| Non carcinogenics | 2.82E−05 | 4.57E−05 | 6.93E−03 |
| Respiratory effects | 9.12E−06 | 2.74E−05 | 9.64E−04 |
| Smog | 7.75E−06 | 7.17E−05 | 1.11E−06 |
| CO₂ eq. (kg) | 4.35E−03 | 0.133 | 0.2.29 |

### Misc

This application claims the priority of U.S. Provisional Application No. 62/960,775 filed Jan. 14, 2020. Additional variations may be apparent to one of ordinary skill in the art from reading U.S. Provisional Application No. 62/960,775, the entire contents of which are incorporated herein by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

It should be appreciated by the person skilled in the art that modifications, additions, or omissions may be made to the methodologies (as well as products thereof) described herein without departing from the scope of the disclosure. For example, the methods described may include more, fewer or other steps. Additionally, steps may be performed in any suitable order.

Although specific advantages or advantageous properties have been enumerated above, various embodiments may include some, none, or all of the enumerated advantages or properties. Other technical advantages may become readily apparent to the person skilled in the art after review of this description and the following figures. It should be understood at the outset that, although exemplary embodiments are illustrated in the figures and described herein, the principles of the present disclosure may be implemented using any number of techniques, whether currently known or not. The present disclosure should in no way be limited to the exemplary implementations and techniques illustrated in the drawings and described herein.

1. A method for biofabricating a biotextile material, the method comprising:
(a) obtaining bacterial cellulose nanofibers;
(b) applying to the bacterial cellulose nanofibers a tanning treatment employing a phospholipid emulsion comprising lecithin; and
(c) processing the cellulose nanofibers into a three-dimensional (3D) shape.

2. The method of claim 1, further comprising subjecting the bacterial cellulose nanofibers to an aldehyde treatment.

3. The method of claim 1, further comprising subjecting the bacterial cellulose nanofibers to a coloration treatment.

4. The method of claim 1, wherein the phospholipid emulsion employed in the tanning treatment applied to the bacterial cellulose nanofibers in (b) further comprises one or more of sunflower seed oil or other phospholipids, fatty acids and choline.

5. The method of claim 1, wherein the bacterial cellulose nanofibers are biologically functional bacterial cellulose nanofibrils produced extracellularly via microbial biosynthesis by living cells of gram-negative bacteria.

6. The method of claim 1, wherein the bacterial cellulose nanofibers are biologically functional and are formed from a culture containing living cells of Gluconacetobacter xylinus bacteria.

7. The method of claim 1, wherein the bacterial cellulose nanofibers are biologically functional and are molded into the 3D shape in (c).

8. The method of claim 1, wherein the bacterial cellulose nanofibers are arranged as a three-dimensional layered structure formed by a self-assembled network of biologically functional bacterial cellulose nanofibrils produced extracellularly via microbial biosynthesis by living cells of gram-negative bacteria.

9. The method of claim 8, wherein the bacterial cellulose nanofibers are arranged as the three-dimensional layered structure in a cultivation vessel having a shape corresponding to the 3D shape.

10. The method of claim 1, wherein the bacterial cellulose nanofibers are arranged as a three-dimensional network of unaligned biologically functional nanofibers in a cultivation vessel having a shape corresponding to the 3D shape, and the phospholipid emulsion is applied in (b) to the biologically functional bacterial cellulose nanofibers in the cultivation vessel, the unaligned biologically functional nanofibers self-assembling into a three-dimensional structure corresponding to the 3D shape.

11. (canceled)

12. (canceled)

13. The method of claim 1, wherein
the bacterial cellulose nanofibers are obtained in (a) via biosynthesis, and
the phospholipid emulsion comprising lecithin is applied in (b) to the bacterial cellulose nanofibers during the biosynthesis.

14. The method of claim 1, wherein
the bacterial cellulose nanofibers are obtained in (a) via biosynthesis, and
the phospholipid emulsion comprising lecithin is applied in (b) to the bacterial cellulose nanofibers after the biosynthesis.

15. The method of claim 1, wherein the cellulose nanofibers are processed into the 3D shape in (c) before, while or after the phospholipid emulsion comprising lecithin is applied in (b) to the bacterial cellulose nanofibers.

16. The method of claim 15, wherein the phospholipid emulsion comprising lecithin is applied in (b) to the bacterial cellulose nanofibers, at least partially at same time that the cellulose nanofibers are being processed into the 3D shape in (c).

17. The method of claim 15, further comprising:
processing the bacterial cellulose nanofibers obtained in (a) into a slurry; and
depositing the slurry comprising the processed bacterial cellulose nanofibers into a cultivation vessel,
the bacterial cellulose nanofibers in the slurry in the cultivation vessel being processed into the 3D shape in (c).

18. The method of claim 15, further comprising:
processing the bacterial cellulose nanofibers obtained in (a) into a slurry; and
depositing the slurry comprising the processed bacterial cellulose nanofibers into a cultivation vessel,
in (b) applying the phospholipid emulsion comprising lecithin to the bacterial cellulose nanofibers in the slurry in the cultivation vessel.

19. The method of claim 15, further comprising:
processing into a slurry the bacterial cellulose nanofibers to which the phospholipid emulsion comprising lecithin has been applied in (b); and
depositing the slurry comprising the processed bacterial cellulose nanofibers into a cultivation vessel,
the bacterial cellulose nanofibers in the slurry in the cultivation vessel being processed into the 3D shape in (c).

20. A biotextile product formed by the method of claim 1.

21. A biofabrication method for forming a biotextile material, the method comprising:
(a) obtaining microbial cellulose nanofibers;
(b) applying a phospholipid emulsion comprising lecithin to the microbial cellulose nanofibers; and
(c) subjecting the microbial cellulose nanofibers to an aldehyde treatment.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A biotextile material comprising biologically functional microbial cellulose nanofibers constituted of biologically functional microbial nanocellulose to which lecithin tanning has been applied, the lecithin tanning applied to the microbial cellulose nanofibers imparting an improved flame retardance property to the biotextile material.

38. (canceled)
